(19) United States
(12) Patent Application Publication
(10) Pub. No.: US 2020/0227172 A1
(43) Pub. Date: Jul. 16, 2020

(54) DETERMINING INDICATORS OF INDIVIDUAL HEALTH

(71) Applicants: Bradley A. Perkins, San Diego, CA (US); Kuan-Fu Ding, San Diego, CA (US); Larry Heminger, San Diego, CA (US)

(72) Inventors: Bradley A. Perkins, San Diego, CA (US); Kuan-Fu Ding, San Diego, CA (US); Larry Heminger, San Diego, CA (US)

(21) Appl. No.: 16/742,593

(22) Filed: Jan. 14, 2020

Related U.S. Application Data
(60) Provisional application No. 62/791,980, filed on Jan. 14, 2019.

(51) Int. Cl. G16H 50/30; G16H 10/60; G16H 20/60; G16H 20/30; G16H 50/70

(52) U.S. Cl. CPC G16H 50/30; G16H 10/60; G16H 50/70; G16H 20/30; G16H 20/60

(57) ABSTRACT

Techniques and systems for determining indicators of the health of individuals are described. The indicators can include scores that help individuals to determine the state of their health. Recommendations with specific service recommendations can also be determined that can help individuals increase the health indicators of the individuals. The techniques and systems described herein leverage the large amounts of medical knowledge, individual health behavioral and environmental data to produce measures of an individual's health status. In implementations, one or more models can be evaluated with respect to health information of the individuals to determine the health scores for the individuals.

### CROSS REFERENCE TO RELATED APPLICATION

This U.S. patent application claims priority to provisional U.S. patent application No. 62/791,980, entitled “DETERMINING INDICATORS OF INDIVIDUAL HEALTH,” and filed on Jan. 14, 2019, the entirety of which is incorporated herein by reference.

### BACKGROUND

The amount of medical knowledge is growing at an increasing rate. The amount of research being conducted by medical scientists along with the use of machine learning and artificial intelligence have resulted in large amounts of new medical knowledge being discovered. By some estimations, the amount of medical knowledge doubled at a rate of every 50 years in 1950, every 7 years in 1980, and every 3.5 years in 2010. It is projected that the rate of doubling of medical knowledge by 2020 will be 0.2 years or about 73 days. (Densen P. “Challenges and opportunities facing medical education.” Transactions of the American Clinical and Climatological Association. 2011; 122:48-58). However, it can take an average of 17 years for research evidence to reach clinical practice. (Morris, Zoe Slote, Steven Wooding, and Jonathan Grant. “The Answer Is 17 Years, What Is the Question: Understanding Time Lags in Translational Research.” Journal of the Royal Society of Medicine 104.12 (2011): 510-520. PMC. Web. 18 Apr. 2018.) Much of the medical knowledge being discovered does not make it to a clinical setting and is unable to benefit individuals or the time needed to bring medical knowledge into the clinical setting takes so long that large numbers of individuals that could have benefited from that medical knowledge are unable to do so.

Additionally, the amount of information accessible to individuals about their health is also increasing. The proliferation of wearable devices, such as smart watches, glucose monitors, and activity trackers, can make information available to individuals about their health on a regular basis. Electronic medical records, improved diagnostic technologies and laboratory testing procedures have also increased the amount of information available to individuals about their health. Further, the increasing availability of genetic testing and the links between certain genes and various health conditions have provided much insight to individuals about their propensity for developing particular health conditions.

Even with the increased amount of medical knowledge and health information available to individuals, there are countless missed opportunities where individuals could have applied this abundance of knowledge to positively impact their health.

### BRIEF DESCRIPTION OF THE DRAWINGS

The Detailed Description is set forth with reference to the accompanying figures, in which the left-most digit of a reference number identifies the figure in which the reference number first appears. The use of the same reference numbers in the same or different figures indicates similar or identical items or features.

FIG. 1 is a diagram of an example environment to determine indicators of the health of individuals.

FIG. 2 is a diagram showing how certain factors can be utilized in conjunction with an individual's health data to determine a health score for the individual, according to some implementations.

FIG. 3 illustrates an arrangement corresponding to different health factors having different amounts of impact on health indicators of different individuals.

FIG. 4 is a block diagram illustrating an example system that includes a number of components of the health science service provider.

FIG. 5 illustrates an architecture to generate one or more models to determine health indicators for individuals.

FIG. 6 illustrates an architecture to utilize health information of individuals associated with an enterprise to determine health indicators of the affiliated individuals and to determine health recommendations for the affiliated individuals.

FIG. 7 illustrates an architecture to identify one or more individuals within a population that represent another individual in a different state of health and/or a different life stage.

FIG. 8 is a block diagram illustrating an example system to determine indicators of the health of individuals.

FIG. 9 is a flow diagram of an example process to determine indicators of the health of individuals.

FIG. 10 is a flow diagram of an example process to determine recommendations to increase health scores of individuals.

FIG. 11 is a flow diagram of an example process to determine recommendations to improve the health scores of individuals based on behavioral characteristics of the individuals

FIG. 12 is a flow diagram of an example process to utilize a model to determine a health indicator for an individual and recommendations for the individual to improve the health indicator.

FIG. 13 is an example user interface including an indicator of the health of an individual.

FIG. 14 is an example user interface including a recommendation to increase a health score of an individual.

FIG. 15 illustrates a framework to determine a health indicator for an individual.

### DETAILED DESCRIPTION

Techniques and systems for determining indicators of individual health are described. The indicators can include scores that help individuals to determine the state of their health. Recommendations can also be determined that can help individuals optimize their health. In particular, the techniques and systems described herein leverage the large amounts of medical knowledge and individual health, behavioral and other (e.g., financial) data to produce measures of an individual's health status.

Large amounts of medical knowledge can be difficult, if not impossible, for an individual or physician or other health professional to analyze and sift through to determine which bits of medical knowledge are applicable to them. Additionally, an individual needs data about their health in order to apply the medical knowledge. Also, data without context and being able to apply the data to an individual's situation/personal health is often not helpful. Systems and techniques described herein aggregate medical knowledge to identify the most important factors that can influence an individual's health and then collect and analyze the individual's health data with respect to those factors in order to determine an indicator of the individual's health. The systems and techniques can also analyze an individual's health data in relation to the existing medical knowledge to determine recommendations that can help to optimize the individual's health.

In various implementations, a health science service provider can obtain health information about an individual from a number of sources. For example, the health information can be obtained from sensors that detect physiological activity, such as heart rate monitors, blood pressure monitors, glucose monitors, body temperature thermometers, combinations thereof, and the like. The sensors can be part of a wearable or implantable biosensor device, such as a watch or an activity tracker. The health information of the individual can also be obtained from laboratory tests and electronic medical records produced by health care providers that have interacted with the individual. Additionally, the health information obtained by the health science service provider can include genetics data. Further, the health science service provider can obtain nutritional information about the individual in additional to physical activity information. In particular implementations, the health science service provider can obtain the health information of the individual via electronic means, such as from mobile devices, wearable and implantable devices, electronic medical records, combinations thereof, and so forth.

The health science service provider can also determine a number of factors that can impact the health of the individual. In various implementations, the health science service provider can analyze medical literature to identify factors that can impact the health of the individual. For example, the health science service provider can analyze academic papers and/or clinical research studies to determine factors that can have an effect on the health of the individual. The health science service provider can also determine factors affecting the health of the individual and validate the importance of these factors in independent data sets before incorporating them into their models. The factors can be validated through one or more independent studies using one or more populations of individuals. In particular implementations, computer-implemented algorithms can be trained using a corpus of health information from a number of populations to validate the factors.

Further, the models utilized to determine health indicators of individuals and recommendations that impact the health indicators can be dynamically updated as new information related to the health of individuals becomes available. In particular, the health science service provider can actively monitor sources of health-related information to identify new research that indicates factors that can impact the health of individuals. The health science service provider can also actively monitor sources of health-related information to identify new interventions and service providers that can impact the health of individuals. In this way, the health science service provider can update the models used to determine the health indicators of individuals and, consequently, the health service provider can more accurately determine the health indicators of the individuals. Further, the health science service provider can recommend certain interventions and service providers that can impact the health of individuals that the individuals and/or their healthcare providers may not otherwise become aware of for several more months or even several more years due to the lack of accessibility of information about the interventions and service providers.

The health science service provider can aggregate the factors affecting the health of the individual into a model. The model can be utilized to determine one or more indicators of the health of the individual. In certain implementations, the indicators of the health of the individual can be represented by one or more scores. The scores can be generated by identifying health information of the individual that corresponds to the factors that can impact the health of the individual. For example, a factor affecting the health of the individual can include blood pressure and the health science service provider can parse the health information of the individual to obtain blood pressure readings for the individual. The health science service provider can also parse health information of the individual for data relating to other factors that can impact the health of the individual, such as cholesterol levels, blood glucose levels, body mass index, level of physical activity, alcohol use, tobacco use, and intake of fruits, vegetable, and nuts. The health science service provider can then apply the data from the individual to one or more models that include factors that can impact the health of the individual. Based on the values of the health information of the individual for the various factors and the amount of impact that each factor can have on the health of the individual, one or more scores can be generated that indicate a level of health of the individual.

The health science service provider can also generate recommendations to optimize the health of the individual. In implementations, the health science service provider can determine a factor impacting the health of the individual that has values that are outside of predetermined levels. The health science service provider can then determine that a change in the value of the factor can increase the score of the individual. In an illustrative example, the health science service provider can determine that the blood pressure of the individual is equal to or above a threshold level. The health science service provider can then provide a recommendation to the individual to lower their blood pressure and identify particular service providers to implement these recommendations which are matched to individuals based on their behavioral preferences, geography, health insurance benefits, socioeconomic status and other data. The health science service provider can also indicate an amount of increase for the health score of the individual based on various improvements in the blood pressure of the individual. Furthermore, the health science service provider can provide recommendations to the individual directed to lowering the blood pressure of the individual. In a particular example, the health science service provider can provide recommendations regarding nutrition and physical activity for the individual to lower their blood pressure and increase their health score. In various situations, the health science service provider can analyze information obtained about the nutrition and activity level of the individual to determine the recommendations for improving the blood pressure and correspondingly increasing the health score of the individual.

The health science service provider can also determine recommendations for individuals based on behavioral profiles of individuals. In these situations, the health science service provider can determine one or more behavioral characteristics of an individual. In certain implementations, the health science service provider can determine indicators of behavioral characteristics of individuals, such as numerical scores corresponding to one or more behavioral characteristics. The behavioral characteristics can correspond to an amount of extroversion, an amount of introversion, an amount of openness to new experiences, an amount of conscientiousness, an amount of emotionality, an amount of agreeableness, an amount of honesty-humility, combinations thereof, and the like. The behavioral characteristics of individuals can be identified based on one or more behavioral evaluations performed with respect to individuals. By determining recommendations for individuals to increase their health scores based on behavioral profiles of individuals, the health science service provider can identify recommendations that have the potential to aid the health of individuals, but that the individuals are also more likely to implement.

Additionally, the techniques and systems described herein can analyze information related to the health of individuals associated with enterprises, such as employees of businesses, to generate recommendations to the employees and to the enterprises that can impact the health of the individuals. In certain situations, enterprises can have access to large amounts of information about the health of their employees through insurance claims and/or participation in employee wellness programs. Typically, the enterprises are unaware of how to analyze this information in a manner that can improve the health of its employees (and customers) and decrease healthcare costs or increase financial value for the enterprises. The health science service provider can analyze data that is related to enterprises and their employees to determine certain interventions that can impact the health of their employees (and customers). For example, the health science service provider can identify certain healthcare providers and/or certain wellness programs that are associated with positive impacts to the health of employees (and customers) of an enterprise and generate recommendations for particular employees of an enterprise to visit the healthcare providers and/or participate in the wellness programs identified by the health science service provider.

By analyzing medical literature and clinical studies and identifying factors that can impact the health of an individual, the health science service provider can provide the individual with medical knowledge that may not have otherwise been available to the individual. Additionally, by applying the individual's specific health information to the various factors that can impact the health of the individual, the health science service provider can generate one or more indicators of the health of the individual that can be straightforward with the meaning of the indicators readily discernible. Furthermore, by generating recommendations to optimize the health of the individual, the health science service provider can help the individual to readily identify specific actions that the individual can take to optimize their health. In these ways, the techniques and systems described herein can reduce the amount of time that medical knowledge takes to be applied to the health of individuals. In addition, the techniques and systems described herein can provide a personalized indicator to individuals about their health and provide practical actions for individuals to improve their health. Thus, individuals do not need to spend countless hours combing the medical literature, which may be difficult to access and understand, to identify factors that affect their health and determine actions that can be taken to optimize their health because the health science service provider can aggregate and analyze medical knowledge in view of individuals' personal health information to generate indicators of the health of individuals. Also, the continual updating of models used to determine health indicators of individuals and used to determine recommendations that can impact the health of the individuals based on the latest scientific research can provide more accurate indicators of an individual's health and provide interventions that are more effective and/or up-to-date than models that are static or updated infrequently.

These and various other example features will be apparent from a reading of the following description and a review of the associated drawings. However, the claimed subject matter is not limited to implementations that solve any or all disadvantages or provide any of the benefits noted in any part of this disclosure.

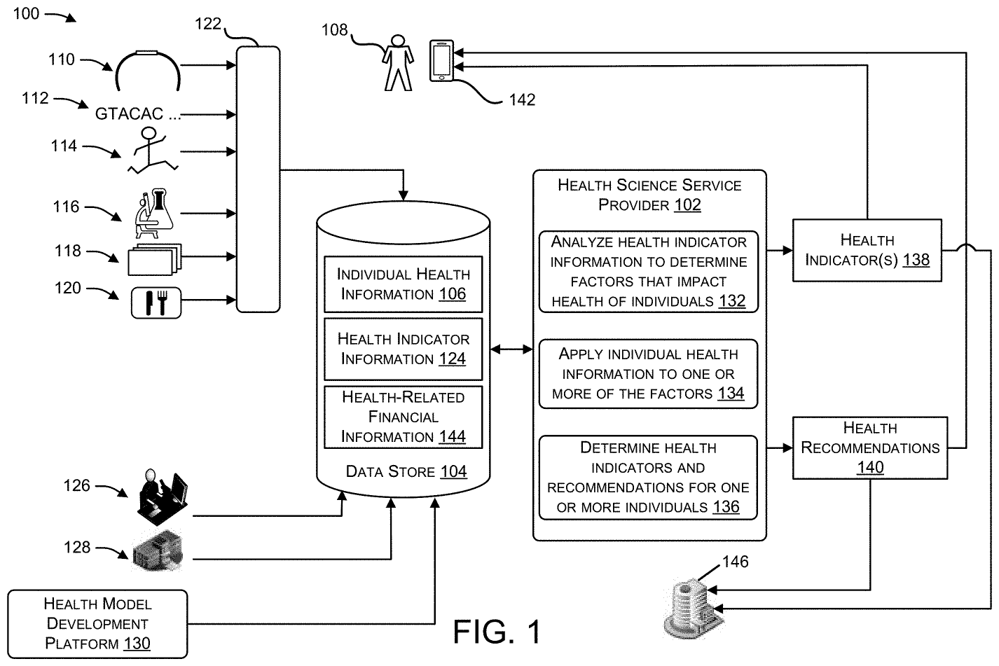

FIG. 1 is a diagram of an example environment 100 to determine indicators of the health of individuals. The environment 100 includes a health science service provider 102 that analyzes information related to the health of individuals to generate indicators of the health of the individuals. The indicators of the health of the individuals can include health scores. In particular implementations, the health science service provider 102 can aggregate and analyze medical literature and other scientific health-related information to determine factors that can affect the health of the individual and apply the personal health information of individuals in conjunction with the factors to determine one or more indicators of the health of individuals.

The health science service provider 102 can include or otherwise access a data store 104. The data store 104 can include one or more non-transitory, tangible computer readable media that stores data, computer-readable instructions, and other information that can be utilized to determine indicators of the health of individuals. In particular implementations, the data store 104 can store individual health information 106. The individual health information 106 can include information that can be utilized to determine the health of individuals. The individual health information 106 can be associated with a number of individuals, such as individual 108. In various implementations, at least a portion of the individual health information 108 can be derived from electronic medical records that are accessible to the health science service provider 102. In certain implementations, the electronic medical records can be accessible by the health science service provider 102 via a third-party repository that stores electronic medical records of individuals. The electronic medical records can be accessed by the health science service provider 102 after obtaining authorization from the individuals that are related to the electronic medical records.

In example implementations, the individual health information 106 can include sensor data 110. The sensor data 110 can include data obtained by sensors that can measure physiological activity and/or physiological characteristics of individuals, or sensors which capture environmental data relevant to individuals (e.g., measurements of air pollution). In particular implementations, the sensor data 110 can be captured by wearable or implantable devices, computing devices, or a combination thereof. In certain examples, the sensor data 110 can indicate heart rate, blood pressure, blood glucose levels, body temperature, environmental data combinations thereof, and the like. In various implementations, the sensor data 110 can be captured with respect to an application executing on a computing device.

The individual health information 106 can also include genetic information 112. The genetic information 112 can indicate the sequence of nucleotides (e.g., monogenic variants or single nucleotides at specific locations (e.g., single nucleotide polymorphisms) that comprise the deoxyribonucleic acid (DNA) of individuals. The genetic information 112 can be obtained by the health science service provider 102 or by a third-party that analyzes blood, tissue and/or saliva samples taken from individuals to determine the makeup of the DNA of the individuals.

In addition, the individual health information 106 can include activity data 114 for individuals. The activity data 114 can indicate types of physical activity in which individuals can participate. For example, the activity data 114 can indicate that individuals are participating in different types of exercise, such as swimming, running, bicycle riding, rowing, yoga, weight training, combinations thereof, and so forth. Additionally, the activity data 114 can indicate participation by the individuals in various sports, such as soccer, tennis, basketball, volleyball, and so forth. The activity data 114 can also indicate times and/or frequency that individuals participate in certain activities in addition to showing the amounts of time that the individuals participate in the activities. In particular implementations, the activity data 114 can be determined based at least partly on the sensor data 110. To illustrate, an increased heart rate in addition to change in location of individuals can indicate that individuals are jogging or taking a walk. In additional implementations, the activity data 114 can be determined based on entries made by individuals into a computing device application, into a log, or into a journal indicating the physical activity of the individuals and an amount of time that the individuals participated in the physical activity. The physical activity data 114 can also be obtained from establishments that conduct classes related to exercise or physical fitness. Data from these activities can be summarized as measures including metabolic equivalent (also known as a “MET”) and related to health benefits. A single MET is defined as the amount of oxygen a person consumes (or energy expended) per unit of body weight during 1 minute of rest.

Further, the individual health information 106 can include medical imaging and laboratory test data 116. Medical imaging data may include techniques using radiation like CT scans or imaging using ultrasound, magnetic resonance imaging or combinations of these methods. These data may include image post-processing, volume and other measurements facilitated by machine learning algorithms. The laboratory test data 116 can indicate levels of certain hormones, proteins, and other molecules that can indicate biological conditions or a level of health of an individual. Laboratory test data may also include DNA sequence information and other means of characterization of the microbiome, the collection of bacteria and other microorganisms which live in and on the human body. The laboratory test data 116 can be obtained from electronic medical records of individuals and/or from third parties that perform tests that measure levels of certain molecules within the body from samples of tissue and/or bodily fluids obtained from individuals. In implementations, the individual health information 106 can include electronic medical records 118. The electronic medical records 118 can include documents from files of physicians and other healthcare providers that have treated or consulted with individuals. The medical records 118 can be obtained from a repository of the healthcare providers after appropriate authorization has been obtained from the individuals to store their medical records in the data store 104.

Additionally, the individual health information 106 can include nutrition information 120. The nutrition information 120 can indicate types of food and beverages consumed by individuals. The nutrition information 120 can also indicate amounts of food and beverages consumed by individuals and times when the food and liquids were consumed. In particular scenarios, the nutrition information 120 can indicate nutrients included in the food and liquids consumed by individuals in addition to calories of the food and beverages. This may include assessment of overall macronutrient proportions (e.g., fat, protein, carbohydrates) in food and beverages and the relationship of these proportion of other physiologic measure like glucose. In various implementations, the nutrition information 120 can be logged by and obtained from an application executing on a computing device that accepts user input indicating the nutrition information. In certain implementations, the nutrition information 120 can indicate a number of servings of certain nutrients and/or servings of various types of nutrients consumed by individuals over a period of time, such as one day, one week, or one month so that this information can be more easily interpreted in context of available scientific studies.

The individual health information 106 can be obtained by the health science service provider 102 and stored in the data store, in certain implementations, via one or more interfaces, such as a representative interface 122. In particular situations, the interface 122 can include a software interface and/or a hardware interface. In additional implementations, the interface 122 can include an application programming interface (API). In certain implementations, the health science service provider 102 can utilize the interface 122 to obtain the individual health information 106 from various entities. For example, the health science service provider 102 can utilize calls, routines, protocols, definitions of the interface 122 to query computing devices to retrieve the individual health information 106. In an illustrative example, the health science service provider 102 can utilize features of an API to retrieve physiological data from wearable or implantable devices of individuals. In an additional example, a third-party platform can aggregate at least a portion of the health information of individuals and the health science service provider 102 can utilize features of an API to retrieve individual health information 106 from the third-party platform. In further implementations, various entities can utilize the interface 122 to send individual health information 106 to the health science service provider 102. For example, the health science service provider 102 can provide an API to access the data store 104 and entities providing at least portions of the individual health information 106 to the data store 104 can utilize features of the API to store the individual health information 106 in the data store 104.

The data store 104 can also store health indicator information 124. The health indicator information 124 can include information that can be utilized to determine factors that can be utilized to generate indicators of the health of individuals. For example, the health indicator information 124 can include clinical trials information 126. The clinical trials information 126 can include information about populations participating in clinical trials, biological conditions being studied during the clinical trials, treatments for the biological conditions, the effectiveness of the treatments for the populations, or combinations thereof. In various implementations, the clinical trials information 126 can be stored in a publicly accessible repository, such as clinicaltrials.gov, presented at scientific meetings, or published in peer-reviewed medical literature. The clinical trials and other available data sets (e.g., Million Veterans Program https://www.research.va.gov/mvp/researchers.cfm) information 126 can also be stored by institutions conducting the clinical trials or making other data available and the institutions can provide access to at least portions of the clinical trials or other health data information 126 to the health science service provider 102.

Additionally, the health indicator information 124 can include medical research information 128. The medical research information 128 can include findings produced by research institutions that indicate treatments for biological conditions, factors that can affect biological conditions, characteristics of populations studied by the research institutions with respect to certain biological conditions, or combinations thereof. In various implementations, the clinical trials information 126 and the medical research information 128 can be included in publications, such as journal articles and/or be made available to other researchers. The publications and/or the data can be publicly or otherwise accessible via certain websites and means of electronic access. The health science service provider 102 can access the websites or other means of electronic access in order to obtain and store at least portions of the clinical trials information 126 and at least portions of the medical research information 128.

The health indicator information 124 can also include information obtained from a health model development platform 130. The health model development platform 130 can provide an online environment that enables software code to be written and executed by developers that is related to identifying factors that affect the health of individuals. In particular implementations, the health model development platform 130 can include reference data sets with phenotype and genetic information, software code segments, software code objects, routines, libraries, combinations thereof, and so forth, that can be utilized to code models that can indicate the health of individuals. The health model development platform 130 can include different versions of algorithms that can be built upon by a number of developers working together to produce an optimized version of one or more algorithms that can indicate the health of individuals. In various examples, the health model development platform 130 can be used to develop algorithms that can determine factors impacting the health of individuals. The health model development platform 130 can also provide access to information that can be utilized to validate models developed with the health model development platform 130. For example, developers using the health model development platform 130 can identify characteristics of populations, clinical trials information, medical research, or combinations thereof that can be utilized to validate algorithms developed via the health model development platform 130.

The health science service provider 102 can continually update the health indicator information 124 by monitoring medical research repositories for new information that can indicate the health of individuals. For example, the health science service provider 102 can monitor attend scientific medical meetings, websites, such as biorxiv.org, and other leading peer-reviewed medical journals, professional society and governmental guidelines to identify additional information that can impact the health of individuals. The health science service provider 102 can implement one or more webcrawlers and/or utilize calls from one or more application programming interfaces (e.g. PubMed, https://www.ncbi.nlm.nih.gov/pubmed/) to identify and extract data from various websites that provide information related to the health of individuals.

In various implementations, the health indicator information 124 can include genetics-related information. For example, the health indicator information 124 can include monogenic variation information. The monogenic variation information can indicate variants of genes in protein encoding regions of DNA. A monogenic variant of a gene can have a single variation in a protein encoding region of DNA of an individual. Monogenic variants can be classified according to an increased or decreased (e.g., certain variation in the gene PCSK9 decrease risk of coronary artery disease) amount of risk that an individual having DNA with the monogenic variant can have with respect to one or more biological conditions. In an illustrative example, monogenic variants can be classified as “benign”, “likely benign”, “uncertain significance”, likely pathogenic”, or “pathogenic” where each classification is associated with a particular range of increased likelihood of an individual with the monogenic variant developing one or more biological conditions. Using the medical literature, it is increasingly possible to derive estimates of relative risk or other similar statistical measures for particular monogenic variants which can be used in conjunction with other quantitative data to establish individual care threshold for early detection and prevention of specific medical conditions. In various implementations, the health indicator information 124 can include at least a subset of monogenic variants. To illustrate, the health indicator information 124 can include monogenic variants having at least a threshold likelihood of individuals with the monogenic variants developing one or more biological conditions.

The health indicator information 124 can also include measures derived from single nucleotide polymorphisms (SNPs). SNPs can include variations in single nucleotides of a region of a gene that can correspond to one or more biological conditions. In particular situations, SNPs can occur outside of protein coding regions of DNA. In some illustrative implementations, the health indicator information 124 can include polygenic risk scores that are derived from multiple SNPs which in isolation may contribute only a small risk but when combined as a polygenic risk score can be used to increase predictive accuracy for health-related risks. To illustrate, a polygenic risk score can indicate a probability that an individual having a particular SNP may develop one or more biological conditions. The polygenic risk score can be based on an effect that an SNP can have on an individual developing one or more biological conditions.

In an illustrative example, at 132, the health science service provider 102 can analyze the health indicator information 126 to determine factors that impact the health of individuals. In various implementations, machine learning techniques can be implemented to determine factors that can affect the health of individuals. Additionally, the factors impacting the health of individuals can be personalized. For example, the health of a first group of individuals can be impacted by a first set of factors and the health of a second group of individuals can be impacted by a second set of factors that includes at least one factor that is different from the first set of factors. In particular implementations, the factors that impact the health of individuals can be based at least partly on the genetic information 112 of the individuals. To illustrate, the health indicator information 126 can indicate that individuals with certain genes can be predisposed to one or more biological conditions. The health science service provider 102 can identify one or more individuals having those genes and determine factors that can affect the health of the one or more individuals based on their genetic predisposition to the particular biological condition(s). This is sometimes referred to in the case of monogenic variation as a mendelian randomization study but this technique is broadly applicable to other genetic and risk factor data.

The health science service provider 102 can also determine factors that can affect the health of individuals based at least partly on other characteristics of the individuals. For example, the health science service provider 102 can determine factors that impact the health of individuals based at least partly on the age of the individuals, the sex of the individuals, nutritional intake of the individuals, certain habits of the individuals, demographics of the individuals, physical activity levels of the individuals, combinations thereof, and so forth. In particular implementations, the health science service provider 102 can analyze the health indicator information 126 and compare characteristics of an individual with factors identified in the health indicator information 126 as impacting the health of individuals. The health science service provider 102 can then determine characteristics of the individual that correspond to characteristics attributable to certain biological, behavioral, environmental, social and financial conditions in the health indicator information 126 to determine factors that can impact the health of the individual.

At 134, the health science service provider 102 can apply individual health information to one or more of the factors. For example, the health science service provider 102 can determine a group of factors that can impact the health of the individual 108. The health science service provider 102 can then obtain portions of the individual health information 106 that correspond to the individual 108 and that are related to the group of factors. To illustrate, the health science service provider 102 can perform a targeted search of the individual health information 106 to identify particular health information associated with the individual 108 and to identify data related to the factors included in the group of factors that can impact the health of the individual 108.

In implementations, the individual health information 106 is stored in the data store 104 utilizing data storage techniques that enable the retrieval of information from the data store 104 efficiently with a minimal use of computing resources. In various implementations, the individual health information 106 can be stored in association with one or more tags or other data identifiers. In particular examples, the individual health information 106 for each individual can be stored in a data structure, such as a data table. In additional examples, the individual health information 106 can be stored in association with links or other identifiers indicating a storage location of data within the data store 104. To obtain health information of the individual 108 from the individual health information 106, the health science service provider 102 can utilize one or more identifiers of the individual 108 within the data store 104 to determine the portions of the individual health information 106 associated with the individual 108. The health science service provider 102 can then utilize various identifiers associated with the factors that can impact the health of the individual 108 to retrieve particular portions of the individual health information 106 corresponding to those factors with respect to the individual 108.

In an illustrative example, the health science service provider 102 can determine that blood glucose level and amount of physical exercise per week are factors that impact the health of the individual 108 based on analyzing characteristics of the individual 108 in conjunction with the health indicator information 126. The health science service provider 102 can then parse the individual health information 106 associated with the individual 108 and retrieve data corresponding to blood glucose levels and amounts of physical exercise of the individual 108 utilizing a search of the individual health information 106 using attributes and/or identifiers associated with blood glucose levels and amounts of physical exercise in the data store 104. After obtaining information for the individual 108 associated with the factors affecting the health of the individual 108, the health science service provider 108 can then evaluate a model or implement one or more algorithms using the portions of the individual health information 106 associated with the individual 108 and the factors impacting the health of the individual 108. Continuing with the previous illustrative example, the health science service provider 102 can apply the blood glucose level data and the amount of physical exercise data of the individual 108 to a model developed to indicate the health of individuals.

At 136, the health science service provider 102 can determine health indicators 138 and health recommendations 140 to increase the health indicators 138 for one or more individuals. The health recommendations 140 may include not only actions that individuals can take to increase the health indicators (e.g., eat 7 servings of fruits and vegetables per day or participate in moderate exercise 3-5 times per week), but also services that can help the individuals take the recommended actions, such as an exercise program or a food delivery program that provides meals to individuals and families.

The health indicators 138 can include one or more health scores that indicate the health of individuals. The health indicators 138 can be determined by applying certain portions of the individual health information 106 of the individuals to the factors impacting the health of the individuals. In various examples, the health indicators 138 can be associated with a scale such that levels of health can be associated with ranges of health indicators 138. For example, a first level of health can be associated with a first range of health scores for overall mortality risk and a second level of health can be associated with a second range of health scores, for example for specific diseases or medical conditions. In particular implementations, increasing health scores can be associated with specific health profiles for example an individual which has had a myocardial infarction (or heart attack) and is seeking to prevent a second occurrence. This is referred to as “tertiary prevention”. Individuals can be characterized as having specific opportunities for primary, secondary and tertiary prevention based on a quantitative estimation of the most important steps they can take to protect and improve their health. Scores derived from machine learning-driven models like this have potential to integrate overall health scores (e.g., 10-year mortality risk) with disease-specific scores (e.g., individual following a heart attack) for an “optimized” health score conveying quantitatively prioritized health actions whatever the current individual health status.

In implementations, multiple health indicators 138 can be determined for an individual. To illustrate, a first health indicator 138 can be determined for one or more first factors and a second health indicator 138 can be determined for one or more second factors. In an illustrative example, a first score can be determined related to certain habits of an individual, such as alcohol use, tobacco use, and seatbelt use and a second score can be determined related to more specific heart disease risk. This allows inclusion of increasingly specific health factors relevant to specific diseases in scores. For example, in the case of coronary artery disease (a leading cause of premature death) it may be important to include the results of a coronary artery calcium using a non-invasive CT scan. In certain implementations, a single health indicator 138 can have multiple components, such as one or more components associated with particular habits, one or more components associated with nutritional intake and physical activity, and one or more components associated with physiological measurements, such as blood pressure, cholesterol levels, blood glucose levels (from static period measurements or continuous glucose monitoring data), imaging data, and environmental data.

The health recommendations 140 can include actions individuals can take to increase health indicators 138 of the individuals. For example, a health recommendation 140 can be related with particular services opportunities related to their behavioral and other preferences and characteristics to an individual increasing their activity level or an individual decreasing intake of certain foods. The health recommendations 140 can also indicate an estimated increase in the health indicators 138 for the individuals if the health recommendations 140 are followed. In various implementations, the health recommendations and specific service opportunities 140 can be provided based on one or more health indicators 138 of individuals corresponding to one or more thresholds. Specific services opportunities related to recommendations can be matched based on efficacy (phase 3) and effectiveness (phase 4) data, behavioral preferences, health insurance coverage, other employer benefits, geographic location, built environment (proximity of parks and safety), socio-economic status, financial data and other data. In certain implementations, the health recommendations and specific services opportunities 140 can be related to thresholds associated with one or more components utilized to determine the health indicators 138. To illustrate, a health recommendation 140 related to decreasing sugar intake can be provided based on blood glucose levels of individuals being above a threshold level. Specific service opportunities to implement this recommendation may include evaluation of local primary care physicians' quality of care data for this condition using deidentified claims data or if the individuals health insurance or self-insured employer covers any number of the new national telemedicine-based services addressing diabetes risk (e.g., Omada, https://www.omadahealth.com/ or Virta, https://www.virtahealth.com/). In another illustrative example, a health recommendation and specific services opportunities 140 related to increasing physical activity levels and increasing intake of fruits and vegetables can be based on an overall health score being below a threshold level may make use of very different resources.

The health indicators 138 and the health recommendations and specific services opportunities 140 can be accessible to individuals via one or more computing devices. In various implementations, the health indicators 138 and/or the health recommendations and specific services opportunities 140 can be accessible using an application executing on a computing device, such as a mobile device app. In additional implementations, the health indicators 138 and/or the health recommendations and specific services opportunities 140 can be accessible via one or more websites. Further, the health science service provider 102 can send the health indicators 138 and/or health recommendations and specific services opportunities 140 to individuals in emails, text messages, multimedia messages, through social media, or combinations thereof. In the illustrative example of FIG. 1, the health science service provider 102 can provide the health indicators 138 and/or the health recommendations and specific services opportunities 140 to a computing device 142 of the individual 108. The health science service provider 102 can follow individuals over time to assess and improve models for recommendations and specific services opportunities 140 by using improvement of scores to continually refine and improve machine learning models.

The health science service provider 102 can also provide updates regarding the health indicators 138 and updates regarding the health recommendations and specific service opportunities 140. For example, the individual health information 106 for individuals can change over time and the health science service provider 102 can analyze the updated individual health information 106 for individuals with respect to the factors that impact the health of the individuals to determine updated health indicators 138 and updated health recommendations and specific service opportunities 140. In various implementations, the health science service provider 102 can periodically analyze the individual health information 106 for individuals with respect to the factors impacting the health of the individuals, such as daily, weekly, monthly, or based on some other period of time. In additional implementations, the health science service provider 102 can monitor the individual health information 106 for individuals to determine a threshold amount of change in the individual health information 106 for various individuals. Based on a threshold amount of change taking place with respect the individual health information 106 associated with factors affecting the health of individuals, the health science service provider 102 can update the health indicators 138 of the individuals. The health science service provider 102 can also generate updated health recommendations and specific service opportunities 140 for the individuals. The health science service provider 102 can make updated health indicators 138 and updated health recommendations and specific services opportunities 140 accessible to individuals via computing devices of the individuals.

In various implementations, the health recommendations and service opportunities 140 can be based on health-related financial information 144. The health-related financial information 144 can correspond to financial resources utilized to obtain certain services, such as participating in one or more wellness programs. The health-related financial information 144 can also correspond to financial resources utilized to provide certain interventions to individuals. For example, the health-related financial information 144 can indicate a cost of a pharmaceutical product that can be used to treat one or more biological conditions, a cost of a diagnostic test to determine whether individuals have developed one or more biological conditions, or a cost of a procedure performed by a healthcare provider to treat individuals that have developed one or more biological conditions. In certain implementations, the health-related financial information 144 can indicate an amount of financial resources needed to have a particular impact on the health of an individual. To illustrate, the health-related financial information 144 can indicate an amount of financial resources needed for an individual's body mass index (BMI) to change from 32 to 25. In other illustrative examples, the health-related financial information 144 can indicate an amount of financial resources to cause a low-density lipoprotein (LDL) cholesterol level of an individual to decrease from 145 mg/dL to 125 mg/dL.

The health science service provider 102 can provide the health indicators 138 and/or the health recommendations and specific services 140 to an enterprise 146. The enterprise 146 can include a business, a university, a governmental agency, or another organization or entity. In various implementations, the health science service provider 102 can provide health recommendations and specific services opportunities 140 for one or more individuals affiliated with the enterprise 146 that can impact the health of the individuals affiliated with the enterprise 146. Individuals affiliated with the enterprise 146 can include employees, contractors, and/or family members of employees or customers of the enterprise 146. In illustrative examples, the health science service provider 102 can recommend that employees of the enterprise 146 having health scores within a certain range participate in a particular wellness program related to nutritional intake and physical activity of the employees. The health science service provider 102 can also indicate an estimated healthcare related cost savings for the enterprise 146 of existing enterprise wellness programs and recommend steps to optimize offers to protect and improve the health of employees, contractors, and/or family members of employees or customers

The health science service provider 102 can also determine the recommendations and specific services opportunities based on behavioral characteristics of individuals. That is, the health science service provider 102 can determine behavioral profiles of individuals based at least partly on one or more behavioral assessments performed with respect to the individuals. The behavioral assessments can be analyzed by the health science service provider 102 to determine behavior characteristics of individuals that can correspond to one or more characterizations of behavior. For example, behavior of individuals can be characterized as extraverted vs. introverted, sensing vs. intuition, thinking vs. feeling, or judging vs. perception. In other examples, the behavior of individuals can be characterized as amounts of honesty-humility, emotionality, extraversion, agreeableness, conscientiousness, or openness to experience. These characteristics can be used by the health science service provider 102 to more effectively match individuals with recommendations and specific services opportunities 140.

Behavioral characteristics of individuals can also be determined based on additional data associated with individuals. In particular implementations, behavioral characteristics of individuals can be identified by the health science service provider 102 analyzing financial data of individuals. Analyzing financial data of individuals can enable the health science service provider 102 to determine behavioral characteristics of individuals based on the buying habits of the individuals. For example, individuals that regularly purchase drinks and food at happy hours may be characterized as extroverted and the health science service provider 102 can provide recommendations to increase the health scores of the individuals that are oriented towards extroverts. In other examples, individuals that rent online movies at home and that eat out at restaurants less than a threshold amount can be characterized as introverted and the health science service provider 102 can provide recommendations to increase the health scores of the individuals that are oriented toward introverts. Further, the health science service provider 102 can determine behavioral characteristics of individuals based on other data, such as usage of mobile phones, usage of one or more mobile device applications, location of individuals, combinations thereof, and so forth.

In addition, the health science service provider 102 can characterize various recommendations and specific services opportunities 140 according to the behavioral characteristics of individuals that are most likely to act upon the respective recommendations and specific services opportunities 140. In particular implementations, the recommendations and specific services opportunities 140 can be characterized by the health science service provider 102 based on behavioral characteristics of individuals that have previously acted upon certain recommendations and specific services opportunities. In illustrative examples, the health science service provider 102 can determine that individuals that are extroverted and open to new experiences are more likely to visit a genetic counselor in an effort to increase their health scores, while individuals that are introverted and not open to new experiences are more likely to utilize self-guided mobile device applications in an effort to increase their health scores. Thus, the health science service provider 102 can utilize behavioral profiles of individuals that indicate behavioral characteristics of the individuals to identify recommendations and specific services opportunities for individuals that can improve their health scores.

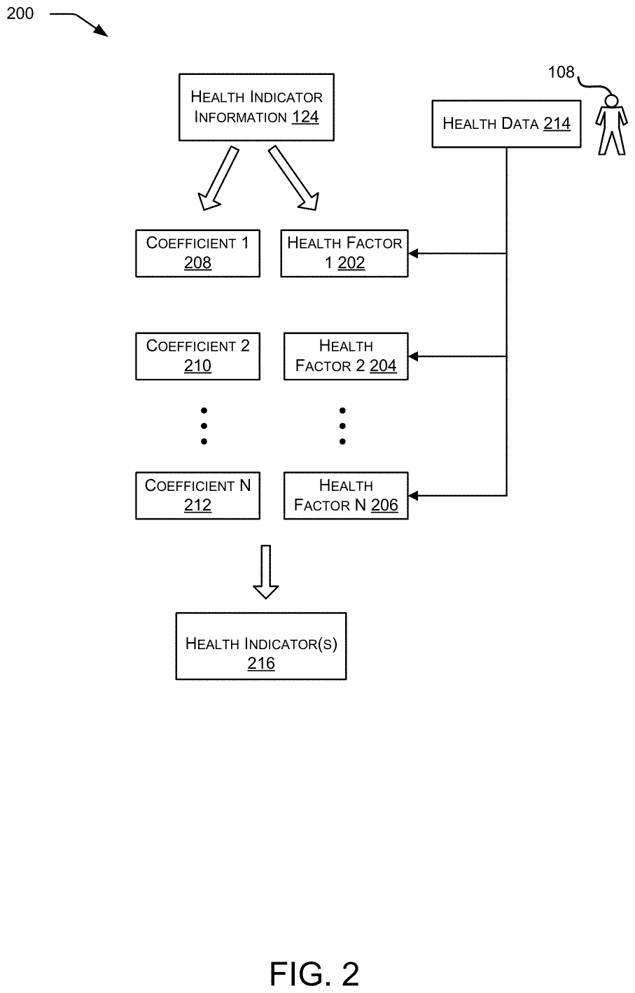

FIG. 2 is a diagram showing how certain factors can be utilized in conjunction with an individual's health data to determine a health score for the individual, according to some implementations. In the illustrative example of FIG. 2, the health indicator information 124 can be utilized to determine a number of health factors that can impact the health of individuals, such as the individual 108. The health factors can apply generally to individuals, the health factors can apply to individuals having certain characteristics, the health factors can apply to specific individuals, or combinations thereof. A health science service provider, such as the health science service provider 102 of FIG. 1, can evaluate the health indicator information 124 to determine the health factors that can impact the health of the individual 108. To illustrate, a health science service provider can determine a first health factor 202 that can impact the health of the individual 108, a second health factor 204 that can impact the health of the individual 108, up to an Nth health factor 206 that can impact the health of the individual 108. In particular implementations, one or more of the health factors 202, 204, to 206 can be genetically related, such as an SNP or a monogenic variant, clinical in character including laboratory or imaging data, behavioral, nutritional, environmental or other.

In additional implementations, the health factors 202, 204, 206 can include health factors that have threshold levels of impact on the health of the individual 108 For example, a health science service provider can utilize one or more statistical techniques to identify health factors that have a statistically significant impact on the health of the individual 108; this may include score reflecting an overall measures of health (e.g., 10-year mortality risk), disease-specific risk (e.g., coronary artery disease), or health optimization using categories of health interventions including but not limited to primary, secondary and tertiary prevention. In particular implementations, the health factors 202, 204, 206 can have a p-value that is greater than a threshold p-value or may improve algorithm performance using measures like area under the curve (i.e., AUC). In certain implementations, the health factors that can impact the health of the individual 108 can change over time. To illustrate, as the health indicator 216 of the individual increases over time, as the individual 108 ages, and/or as other characteristics or habits of the individual 108 change over time, the factors impacting the health of the individual 108 can also change. Furthermore, additional medical research and/or additional clinical trials or other available data and information can cause the factors impacting the health of the individual 108 to be modified. In particular implementations, a health science service provider can re-evaluate the factors having an impact on the health of the individual 108 and update the factors that can have an impact on the health of the individual as new information becomes available and meets certain standards of medical evidence.

The health factors 202, 204, 206 used to evaluate the health of the individual 108 can be part of a model that is generated by the health science service provider 102. To determine health factors that impact the health of individuals, the health science service provider 102 can determine an amount of correlation between health of individuals and a health factor. An amount of correlation between a health factor and the impact of the health factor on the health of individuals can correspond to a probability of a relationship existing between the health factor and the impact of the health of individuals. The health science service provider 102 can also determine an amount of causation (rather than correlation) between health factors and the health of individuals. An amount of causation between a health factor and an amount of impact of the health factor on the health of individuals can correspond to a probability that a biological condition or health related condition is directly attributable to a health factor. In some cases, correlation may not be indicative of causation. The health science service provider 102 can add health factors to a model to evaluate the health of individuals based on at least one of an amount of the causation being at least a threshold amount or an amount of the correlation being at least a threshold amount.

An amount of causation and/or an amount of correlation between a health factor and the impact of the health factor on the health of individuals can be determined by performing an analysis of data that is related to the health factor and one or more biological conditions or one or more health related conditions. The analysis can be performed using one or more machine learning algorithms to determine an amount of causation and/or an amount of correlation between a health factor and its impact on the health of individuals. In certain implementations, the analysis can be performed by the health science service provider 102. In additional implementations, the analysis can be performed by entities other than the health science service provider 102. In these scenarios, the health science service provider 102 can access the analysis performed by the other entities and add one or more health factors to a model to evaluate the health of individuals based on the analysis by the other entities. In certain implementations, the health science service provider 102 can perform an additional analysis with respect to health factors identified by an additional entity that correspond to a biological condition. That is, the health science service provider 102 can perform the additional analysis to verify that the health factor identified by the additional entity does correspond to the biological condition. In various implementations, the health science service provider 102 can utilize data from sources that are different from the data sources utilized by the additional entity in the additional analysis to verify that the health factor identified by the additional entity corresponds to the biological condition. The health factor may not be used by the health science service provider 102 to evaluate the health of individuals in situations where the additional analysis performed by the health science service provider 102 determines that the health factor identified by the additional entity does not correspond to the biological condition.

The health indicator information 124 can also be utilized to determine coefficients that indicate a relative importance (e.g., relative risk) of the health factors in impacting the health of the individual 108. In various implementations, the coefficients can be determined based at least partly on an analysis of information from one or more medical research studies and/or one or more clinical trials indicating an amount of impact that certain factors have on the health of individuals. For example, based on analyzing the health indicator information 124, a health science service provider can determine that different health factors can have different impacts on the health of the individual 108 and the coefficients of the health factors can indicate the varying amounts of impact that the health factors can have on the health of the individual 108.

The coefficients can also be based at least partly on characteristics of the individual 108. For example, the impact of various health factors on the health of individuals can be based at least partly on particular genetic characteristics of the individuals, particular biological characteristics of the individuals, and/or certain nutritional or physical activity habits of the individuals. Thus, the coefficients of the health factors associated with an individual can be related to these characteristics of the individuals. In an illustrative example, individuals having certain genetic traits can be more impacted by certain dietary habits than other individuals. In another illustrative example, blood pressure level can be a factor having a greater impact on individuals with relatively high blood pressure levels. In the illustrative example of FIG. 2, a first coefficient 208 corresponds to the first health factor 202, a second coefficient 210 corresponds to a second health factor 204, and an Nth coefficient 212 corresponds to the Nth health factor 206. Changes to the health of the individual 108 and/or changes to the health-related knowledge can result in changes to the coefficients 208, 210, 212. In certain implementations, the health factors 202, 204, 206 and the coefficients 208, 210, 212 can be included in a model utilized to determine the health of individuals.

Health data 214 of the individual 108 can be utilized to determine one or more health indicators 216 for the individual 108. To illustrate, the particular values of the physiological measurements, medical imaging, and/or habits of the individual 108 that correspond to the factors 202, 204, 206 can be utilized to determine the one or more health indicators 216 in conjunction with the coefficients 208, 210, 212. In an illustrative example, the first health factor 202 can correspond to an amount of physical activity of the individual 108, the second health factor 204 can correspond to blood pressure levels of the individual 108, and the Nth health factor 206 can correspond to medical imaging results of the individual 108. In this scenario, the information related to these factors 202, 204, 206 can be extracted from the health data 214 to determine the health indicators 216. For example, the amount of physical activity of the individual 108, the blood pressure levels of the individual 208, and the cholesterol levels of the individual 108 can be obtained from the health data 214 and evaluated with respect to the coefficients 208, 210, 212 to determine the one or more health indicators 216. In a particular example, a first value can be determined for the first factor 202 and weighted according to the first coefficient 208, a second value can be determined for the second factor 204 and weighted according to the second coefficient 210, and a third value can be determined for the Nth factor 206 and weighted according to the third coefficient 212. In various implementations when a factor is genetic, applying the health data 214 to the factor can produce a monogenic variant or polygenic risk score that can be used to determine one or more of the health indicators 216. The results of the weighting of the factors 202, 204, 206 according to the coefficients 208, 210, 212 can produce the one or more health indicators 216. In various implementations, the one or more health indicators 216 can include one or more health scores.

Over time, health factors added to a model to determine the health indicators 216 can be determined through a machine learning analysis of additional data obtained from a population of individuals. In addition, health factors added to an evaluation of the health of the individual 108 can be determined based on additional medical literature and/or additional clinical data. In particular implementations, health factors that are candidates to be added to a model to determine the health indicators 216 can be evaluated with respect to data that can be used to validate the impact of the candidate factors on the health of individual. The machine learning analysis can determine that the factors to be added have an impact on the health of the individual 108 that is greater than a threshold impact.

Additionally, the health data 214 of the individual 108 can also change over time. As the health data 214 of the individual 108 changes over time, the one or more health indicators 216 can also change over time. In certain implementations, by tracking changes to the health indicator(s) over time, the individual 108 and/or the health science service provider 102 can identify nutritional intake, physical activity, and/or other factors that cause changes in the one or more health indicators 216. For example, as blood glucose level changes in the individual 108 cause changes to the health indicator(s), the individual 108 and/or the health science service provider 102 can identify the foods that result in changes to the health indicator(s). Additionally, to account for outliers in the health data 214, the health science service provider 102 can generate a moving average for one or more portions of the health data 214. In this way, the health science service provider 102 can generate an average value for one or more portions of the health data 214 over a period of time and utilize the average value to determine the health indicator(s) 216.

Further, the candidate health factors can be determining based on different data sources depending on the amount of data available from the various data sources. To illustrate, initially, the health science service provider 102 can store less than a threshold amount of information specific to the individual 108 to determine candidate health factors and/or to analyze certain health factors 202, 204, 206. In these situations, the health science service provider 102 can determine the one or more health indicators 216 based on a combination of health information 124 and health data 214 of the individual 108 and additional health information 124 and health data for a population of individuals that corresponds to the individual 108. For example, the population of individuals used to determine candidate health factors and/or to evaluate the health factors 202, 204, 206 can have a biological profile, a genetic profile, and/or a behavioral profile that corresponds to the biological profile, the genetic profile, and/or the behavior profile of the individual 108. In addition, as more health data 214 and/or health information 124 specific to the individual 108 increases, the candidate health factors and/or the one or more health indicators 216 can be determined by a greater amount of the information associated with the individual 108 and less of the information associated with the population.

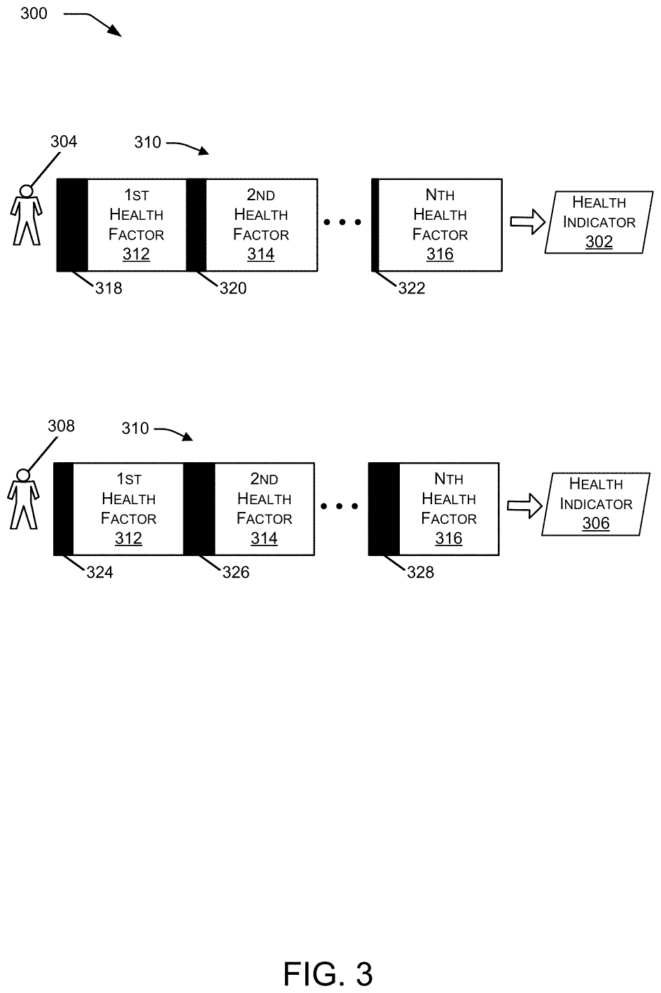

FIG. 3 illustrates an arrangement 300 corresponding to different health factors having different amounts of impact on health indicators of different individuals. The arrangement 300 indicates a first health indicator 302 for a first individual 304 and a second health indicator 306 for a second individual 308. The health indicators 302, 304 can be determined using a model 310 that includes a number of components, such as the first health factor 312, the second health factor 314, up to the Nth health factor 316. The health factors 312, 314, 316 can correspond to one or more biological, behavioral or environmental conditions that can be present in individuals, such as the individuals 304, 308.

The health factors 312, 314, 316 can be determined by the health science service provider 102 as having at least a threshold impact on the health of individuals, including the individuals 304, 306. In particular implementations, the health science service provider 102 can determine that the health factors 312, 314, 316 have at least a threshold amount of causation with respect to the health of the individuals 304, 306. In addition, the health science service provider 102 can determine that the health factors 312, 314, 316 have at least a threshold amount of correlation with respect to the health of the individuals 304, 306. In various implementations, the amount of causation of the health factors 312, 314, 316 with respect to one or more biological conditions can correspond to at least the amount of correlation of the health factors 312, 314, 316 with respect to the one or more biological, behavioral and environmental conditions. That is, a probability that the health factors 312, 314, 316 are directly causative of one or more biological, behavioral or environmental conditions is at least a probability that the health factors 312, 314, 314 are correlated to the one or more biological, behavioral or environmental conditions.

The individual health factors 312, 314, 316 can have a particular impact on the health of an individual. The impact of a health factor on the health of an individual can be expressed as a weighting of the health factor in determining the health indicator for the individual. The weighting of particular health factors on the health indicator for an individual can be different for different individuals. For example, a weighting of a health factor for the first individual 304 can be different than the weighting of a health factor for the second individual 308. The weightings of various health factors can be based at least partly on values associated with the health factors. To illustrate, a weighting for a blood pressure health factor can be greater in situations where an individual is found to be at higher genetic risk based on a polygenic risk score for ischemic stroke, a serious health condition associated with elevated blood pressure. Weightings of health factors can be based at least partly on genetics of an individual. In an illustrative example, an individual can be more disposed toward certain biological conditions based on their genes. Further, weightings of health factors can be based on family history with respect to a biological condition. In a non-limiting example, an individual with a family history of diabetes can have a higher weighting for a blood glucose health factor than an individual without a family history of diabetes.

In the illustrative implementation of FIG. 3, the health indicator 302 for the first individual 304 is determined with respect to a first weighting 318 of the first health factor 312, a second weighting 320 of a second health factor 314, and a third weighting 322 of a third health factor 316. Additionally, the health indicator 306 for the second individual 308 is determined with respect to a fourth weighting 324 of the first health factor 312, a fifth weighting 326 of the second health factor 314, and a sixth weighting 328 of the third health factor 316. In the particular illustration of FIG. 3, the first weighting 318 for the first health factor 312 of the first individual 304 is greater than the fourth weighting 324 for the first health factor 312 of the second individual 308. In addition, the second weighting 320 for the second health factor 314 of the first individual 304 is less than the fifth weighting 326 for the second health factor 314 of the second individual 308. Further, the third weighting 322 for the third health factor 316 of the first individual 304 is less than the sixth weighting 328 for the third health factor 316 of the second individual 308. In various implementations, the first health indicator 302 of the first individual 304 can be a different value than the second health indicator 306 of the second individual 308. In additional implementations, the first health indicator 302 of the first individual 304 can be a same value as the second health indicator 306 of the second individual 308. That is, even though the weightings 318, 320, 322 used to determine the first health factor 302 may be different from the weightings 324, 326, 328 used to determine the second health factor 306, the first health indicator 302 and the second health indicator 306 may be the same or substantially the same.

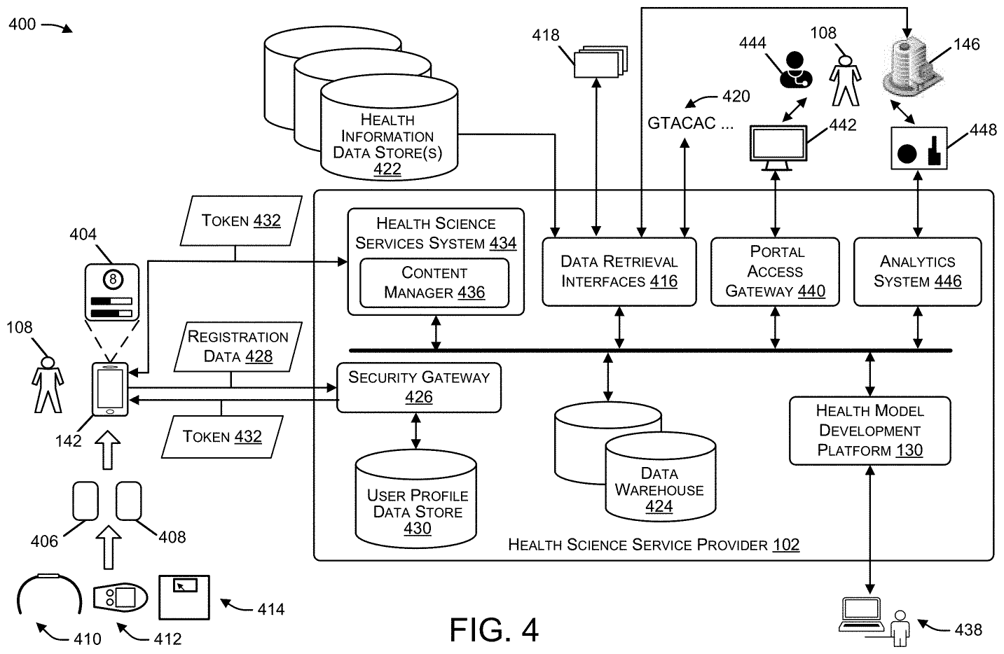

FIG. 4 is a block diagram illustrating an example system 400 that includes a number of components of the health science service provider 102. The health science service provider 102 can analyze data from a number of different sources to determine health indicators, such as scores, for individuals that corresponds to a level of health of the individuals. The health science service provider 102 can utilize one or more models to determine the health indicators for individuals. In certain implementations, the one or more models can be obtained from health-related information resources. For example, the one or more models can include a model that is generated by a research institution based on clinical data. The one or more models can also be obtained from analyzing one or more corpuses of data using machine learning techniques to identify health factors that can impact the health of individuals.

In the particular implementation of FIG. 4, the health science service provider 102 can develop a health science application 404 that can be executed on the computing device 142 of the individual 108. The health science application 404 can display one or more health indicators of the individual 108. The health science application 404 can also display recommendations to increase the one or more health indicators of the individual 108. In addition, the health science application 404 can obtain health related information from one or more additional applications executed by the computing device 142 and send the health-related information to the health science service provider 102. In the illustrative example of FIG. 4, the computing device 142 is executing a first health related application 406 and a second health related application 408. The first health related application 406 and the second health related application 408 can obtain data from one or more health monitoring devices, such as a first health monitoring device 410, a second health monitoring device 412, and a third health monitoring device 414. The health devices 410, 412, 414 can include a fitness tracker, a heart rate monitor, a blood pressure monitor, a blood glucose monitor, a digital scale, a body temperature monitor, a respiratory rate monitor, oxygen level monitors, medical imaging, environmental data combinations thereof, and the like. The first health related application 406 and the second health related application 408 can cause the computing device 142 to display information based on the data obtained from the health monitoring devices, such as a current blood glucose level of the individual 108 and/or a blood glucose level of the individual 108 over a period of time. The health science application 404 can obtain data from the health-related applications 406, 408 using calls of one or more application programming interfaces of the health-related applications 406, 408.

The health science service provider 102 can also obtain health information related to the individual 108 from a number of additional sources. For example, the health science service provider 102 can include one or more data retrieval interfaces 416 that obtain health information of the individual 108. The one or more data retrieval interfaces 416 can obtain health information of the individual 108 using calls of APIs associated with a first data source 418 and a second data source 420. The one or more data retrieval interfaces 416 can also retrieve information from one or more health information data stores 422.

The data retrieval interfaces 416 can also obtain information from the enterprise 146. In particular implementations, the enterprise 146 can provide information indicating individuals that are affiliated with the enterprise 146, such as health-related information of employees. For example, the enterprise 146 can provide health insurance claims information for employees of the enterprise 146 to the health science service provider 102 and costs related to the health insurance claims for employees of the enterprise 146. Additionally, the enterprise 146 can provide information to the health service provider 102 indicated healthcare providers that treat employees of the enterprise 146 and interventions obtained by employees of the enterprise 146. Further, the enterprise 146 can provide information to the health science service provider 102 related to health assessments of employees of the enterprise 146 and information related to wellness programs offered by the enterprise 146. In certain implementations where the enterprise 146 provides goods and/or services to consumers, the health science service provider 102 can obtain information from the enterprise 146 that corresponds to goods and/or services obtained by consumers from the enterprise 146.

In various implementations, the first data source 418 can store electronic health records of the individual 108. The electronic health records can include health records from physicians, hospitals, clinics, health treatment facilities, laboratory testing services, insurance entities, combinations thereof, and the like. In certain implementations, the health science service provider 102 can obtain at least a portion of the electronic health records of the individual 108 from one or more computing devices of the individual 108.

Additionally, the second data source 420 can store genetic information corresponding to the individual 108. The genetic information can include at least a portion of the deoxyribonucleic acid (DNA) sequence of the individual 108. The genetic information can also indicate the presence or absence of certain sequences of DNA that can correspond to the presence of one or more biological conditions. The genetic information of the individual 108 can be obtained from one or more computing devices of the individual 108. Further, the genetic information of the individual 108 can be obtained from an online service that obtains genetic information of individuals. In particular implementations, the data retrieval interfaces 416 can obtain data from publicly accessible data stores (not shown) that store information related to clinical trials and/or clinical research pertaining to the health of individuals. In certain implementations, the clinical trials and/or clinical research can include one or more models to predict biological conditions with respect to individuals.

The health information data stores 422 can include information that can indicate one or more factors that correspond to one or more biological conditions. For example, the health information data stores 422 can store information indicating risk factors for developing one or more biological conditions. The risk factors can be genetic, in some situations. Genetic information can be retrieved from various websites, such as Genopedia: phgkb.cdc.gov/PHGKB/startPagePedia.action, Phenopedia: phgkb.cdc.gov/PHGKB/startPagePhenoPedia.action, and GeneMania: genemania.org/. Additionally, the risk factors can be behavioral. In other scenarios, the risk factors can correspond to biological characteristics of individuals, such as levels of one or more substances associated with an individual (e.g., blood glucose levels, cholesterol levels, levels of various proteins, etc.). Further, the risk factors can be related to nutrition and can include amount of fruit consumed per day, amount of vegetables consumed per day, amount of legumes consumed per day, amount of whole grains consumed per day, amount of nuts and seeds consumed per day, amount of milk consumed per day, amount of red meat consumed per day, amount of processed meat consumed per day, amount of fiber consumed per day, amount of calcium consumed per day, amount of omega 3 fatty acids consumed per day, amount of polyunsaturated fatty acids consumed per day, and amount of trans fatty acids consumed per day. In various implementations, the health information data stores 422 can store information that indicates financial costs related to healthcare. To illustrate, the health information data stores 422 can store information that indicates financial costs for certain treatments of biological conditions. The health information data stores 422 can also store financial costs of services provided by healthcare providers, financial costs of diagnostic tests, and/or financial costs of medications that can be provided for one or more biological conditions.

In certain implementations, the health information data stores 422 can store information that can be used to validate one or more models generated by the health science service provider 102. For example, the health information data stores 422 can store information that was not used to directly generate one or more models by the health science service provider 102. In this way, models generated by the health science service provider 102 can be independently verified. In some situations, the health information data stores 422 can store disease specific data that can be used to validate one or more models generated by the health science service provider 102. Additionally, the health information data stores 422 can store non-disease specific data that can be used to validate one or more models generated by the health science service provider 102. In particular implementations, one or more factors identified from the health information data stores 422 can be added to one or more models of the health science service provider 102 when the factors have a threshold amount of positive impact on the one or more models. To illustrate, an area under a receiver operating characteristic (ROC) curve can be generated when a factor is added to the model. In situations where the area under the operation curve changes by more than a threshold amount, the factor can be added to the one or more models. In various illustrative examples, a threshold increase in the area under the ROC curve can be at least 0.05, at least 0.1, at least 0.2, or at least 0.3.

The information obtained by the health science service provider 102 that is associated with the health of the individual 108 can be stored in a data warehouse 424. In particular implementations, the data warehouse 424 can store information obtained from the health science application 404. The data warehouse 424 can also store electronic health records of individuals, models used to generate health indicators of individuals, and data used to validate the models. Additionally, the data warehouse 424 can store information obtained from the data sources 418, 420. The health science service provider 102 can obtain data from the health science application 404 and/or from data sources via the data retrieval interfaces 416 in a number of formats, such as a comma separated values (CSV) format and a Java script object notation (JSON) format and store the information according to a data storage schema of the health science service provider 102.

The health science service provider 102 can also include a security gateway 426 to control access to information stored by the health science service provider 102. The security gateway 426 can also control access to information generated by the health science service provider 102, such as health indicators of individuals determined by the health science service provider 102. The security gateway 426 can authenticate users before the users can access data stored by and/or generated by the health science service provider 102. In various implementations, the security gateway 426 can generate tokens that enable users to access data stored by and/or generated by the health science service provider 102.

In particular implementations, the computing device 142 can send the registration data 428 to the security gateway 426 to access information stored and/or generated by the health science service provider 102. The registration data 428 can be generated by the health science application 404. In certain implementations, the registration data 428 can be generated based at least partly on one or more identifiers of the individual 108. The one or more identifiers of the individual 108 can include a login identifier of the individual 108, a user name of the individual 108, a password of the individual 108, combinations thereof, and so forth. In particular implementations, the registration data 426 can include a request to access data of the individual 108 stored by the health science service provider 102. In additional implementations, the registration data 428 can include a request to access one or more health indicators of the individual 108 generated by the health science service provider 102.

The security gateway 426 can authenticate the individual based on the registration data 428 and data stored in the user profile data store 430. In various implementations, the security gateway 426 can determine that portions of the registration data, such as one or more identifiers of the individual 108, correspond to information stored by the user profile data store 430 of a user of the services provided by the health science service provider 102. The user profile data store 430 can store information about individuals that have access to information stored by and/or generated by the health science service provider 102, such as email addresses of the individuals, identifiers of the individuals, account information of the individuals, combinations thereof, and the like. The security gateway 426 can generate a token 432 that enables the computing device 142 to access information associated with the individual 108 that is stored and/or generated by the health science service provider 102. In illustrative implementations, the token 432 can include a string of characters that is generated using a random number generator or a pseudo-random number generator. The token 432 can be associated with the individual 108 in the user profile data store 430. In particular implementations, the security gateway 426 can generate the token 432 based on authenticating the individual 108 by determining that information included in the registration data 428 corresponds to information about the individual 108 stored by the user profile data store 430.

The computing device 142 can send the token 432 to a health science services system 434 to access information stored by and/or generated by the health science service provider 102. The health science services system 434 can access data stored by at least one of the data warehouse 424 or the user profile data store 430 and provide the data to the computing device 142 via the health science application 404. The health science services system 434 can include a content manager 436 that generates user interface data for displaying information stored by and/or generated by the health science service provider 102. In various implementations, the content manager 436 can format and arrange information stored by and/or generated by the health science service provider 102 for display via the health science application 404. In particular implementations, the health science services system 434 can obtain or generate one or more health indicators of the individual 108 generated by the health science service provider 102 and the content manager 436 can send user interface data to the health science application 404 that the health science application 404 can use to generate one or more user interfaces that include the one or more health indicators of the individual 108. Additionally, the health science services system 434 can obtain or generate one or more recommendations related to the health of the individual 108. The content manager 436 can send user interface data to the health science application 404 that the health science application can use to generate one or more user interfaces that include the one or more recommendations.

The health science service provider 102 can also include the health model development platform 130. The health model development platform 130 can provide an online environment that enables software code to be written and executed by individuals, such as developer 438, that is related to identifying factors that affect the health of individuals. The health model development platform 130 can be accessible to individuals affiliated with the health science service provider 102, such as employees and/or contractors of the health science service provider 102. Additionally, the health model development platform 130 can be accessible to developers and data scientists outside of the health science service provider 102. The health model development platform 130 can provide an architecture that enables a community to collaborate on developing and validating models that generate indicators of the health of individuals.

Additionally, the health science service provider 102 can include a portal access gateway 440 that can provide online access to information stored by and/or generated by the health science service provider 102. One or more computing devices, such as the computing device 442, can retrieve information via the portal access gateway 440. The portal access gateway 440 can generate one or more user interfaces that display information stored by and/or generated by the health science service provider 102. In particular implementations, the portal access gateway 440 can generate user interfaces for a number of different entities, such as healthcare providers 444, individuals (e.g., individual 108), and/or the enterprise 146. The healthcare providers 444 can include physicians, nurses, physical therapists, laboratory technicians, diagnostic technicians, combinations thereof, and so forth. The enterprises can include insurance companies, healthcare systems, government agencies, combinations thereof, and the like. In various implementations, the portal access gateway 440 can generate different user interface portals for different types of entities. For example, the portal access gateway 440 can provide a first portal for physicians 444, a second portal for the individual 108, and a third portal for the enterprise 146. In certain implementations, the portal access gateway 440 can provide one or more portals to configure profiles for users of the health science service provider 102.

Further, the health science service provider 102 can include an analytics system 446 that can analyze data stored by and/or generated by the health science service provider 102 and provide one or more data analytics user interfaces 448 to the entities 444, 108, and 146. The analytics system 446 can aggregate data of certain groups of individuals or entities and provide the aggregated data via the data analytics user interface 448. In particular implementations, data can be aggregated based on queries provided by the entities 444, 108, 146. In certain implementations, the analytics system 446 can aggregate data that has been collected over a period of time and provide the time-based data via the data analytics user interface 448. In additional implementations, the analytics system 446 can identify trends in data and provide the trends via the data analytics user interface 448. Also, the analytics system 446 can determine key performance indicators for one or more of the entities 444, 108, 146 and determine values for the key performance indicators. The analytics system 446 can provide the values of the key performance indicators via the data analytics user interface 448.

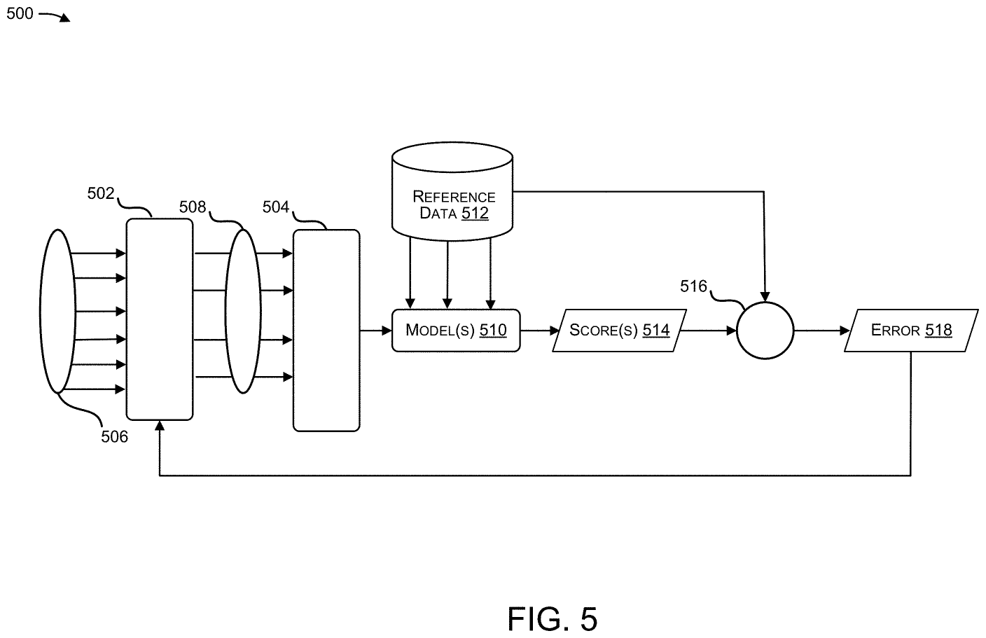

FIG. 5 illustrates an architecture 500 to generate one or more models to determine health indicators for individuals. The architecture 500 includes a first neural network 502 and a second neural network 504. A first set of inputs 506 can be provided to the first neural network 502. The first set of inputs 506 can include data related to the health of a number of individuals. In various implementations, the first set of inputs 506 can include medical records of individuals, laboratory test results of individuals, diagnostic test results of individuals, imaging data, physical activity data of individuals, nutritional information of individuals, vital signs of individuals, other behavioral and environmental data combinations thereof, and the like. In particular implementations, the first neural network 502 can determine an amount of correlation between health-related data for individuals and one or more biological conditions. For example, the first neural network 502 can determine a correlation between values for certain diagnostic tests and one or more biological conditions of individuals. In another example, the first neural network 502 can also determine a correlation between physical activity of individuals and one or more biological conditions.

In illustrative implementations, the first neural network 502 can include a radial basis function (RBF) neural network. The first neural network 502 can include a number of input layers to obtain information from the first set of inputs 506. The first neural network 502 can also include a number of hidden layers to analyze the information from the first set of inputs 506. Additionally, the first neural network 502 can include a number of output layers to provide one or more outputs of the first neural network 502.

In particular implementations, the outputs of the first neural network 502 can be a second set of inputs 508 for the second neural network 504. The second neural network 504 can determine an amount of causation between health-related information of individuals and one or more biological conditions. To illustrate, the second neural network 504 can determine an amount of causation between the intake of foods having certain nutritional content and cardiovascular disease. In various implementations, an amount of causation between certain health related information and one or more biological conditions can be different from an amount of correlation between the certain health related information and the one or more biological conditions.

In illustrative implementations, the second neural network 504 can include a multilayer perceptron (MLP) neural network. The second neural network 504 can include a number of input layers to obtain information from the second set of inputs 508. The second neural network 504 can also include a number of hidden layers to analyze the information from the second set of inputs 508. Additionally, the second neural network 504 can include a number of output layers to provide one or more outputs of the second neural network 504, such as one or more factors to include in one or more models 510. In particular implementations, the second neural network 504 can output factors that were not previously determined to be causative and/or correlated to the health of individuals. In this way, the first neural network 502 and the second neural network 504 can identify new factors that have significant effects on the health of individuals.

The one or more models 510 can include one or more factors with each factor having at least one coefficient. The one or more factors and the respective coefficient(s) of the one or more factors can be determined by at least one of the first neural network 502 and the second neural network 504. The one or more models 510 can be evaluated to determine indicators of health for individuals. Data related to the health of individuals that corresponds to the one or more factors of the one or more models 510 can be provided and evaluated by the one or more models to determine the indicators of health for the individuals. For example, in situations where a model 510 has a blood glucose level factor, data indicating blood glucose levels of the individuals can be evaluated by the model 510.

The one or more models 510 can be validated with respect to reference data 512. The reference data 512 can include data of individuals that have previously been evaluated for certain biological conditions. Additionally, the reference data 512 can include information that corresponds to at least a portion of the factors included in the one or more models 510. In particular implementations, the reference data 512 can also include health scores of individuals that have previously been generated. As part of the validation process, the one or more models 510 can generate one or more scores 514 with respect to the reference data 512. The one or more scores 514 can be analyzed by a function 516, such as a comparator function, with respect to the reference data 512 to determine an amount of error 518 in the scores 514. The amount of error 518 is fed back into the first neural network 502. By providing the first neural network 502 with the amount of error 518, the accuracy of each iteration of the one or more models 510 can improve. Further, as the amount of reference data 512 increases, the accuracy of the one or more models 510 can also increase.

In various implementations, the accuracy of the one or more models 510 can increase with respect to a particular individual as the amount of data obtained for the individual increases. For example, if data is unavailable for an individual for a number of factors included in the one or more models, the score(s) generated for the individual may not be as accurate as for situations where information related to a greater number of factors is available for individuals. In addition, as more information is obtained for individuals over time, the accuracy of the score(s) for the individuals can also increase. To illustrate, blood pressure of an individual can be more accurately determined after blood pressure values of the individual have been obtained for at least a threshold period of time. In this way, a more accurate indication of the health of individuals can be determined not only as the models 510 used to generate the health scores of individuals are improved, but also as the state of the health of the individual is more fully determined as different, additional information becomes available for the individuals and as data collected over at least a threshold period of time is obtained.

In various implementations, the first neural network 502 and the second neural network 504 can determine recommendations with specific service opportunities that individuals can follow that impact the health indicators of the individuals. In these situations, the one or more models 510 can indicate whether or not one or more recommended actions performed by individuals are correlative and/or causative with respect to increasing health indicators of individuals. The one or more models 510 can be evaluated with respect to recommendations with specific service opportunities as data is obtained over time related to the recommendations. Thus, as more data is obtained by the health science service provider 102 about actions that individuals take with respect to certain recommendations with specific service opportunities provided by the health science service provider 102, the one or more models 510 can be updated to reflect increasing or decreasing amounts of correlation and/or causation with respect to the recommendations. In particular implementations, the first neural network 502 and the second neural network 504 can determine that one or more first recommendations that, when performed by individuals, have a statistically significant amount of impact on the health indicators of the individuals. The first neural network 502 and the second neural network 504 can also determine that one or more second recommendations that, when performed by individuals, do not have a statistically significant amount of impact on the health indicators of individuals. The health science service provider 102 can then provide the recommendations with specific services opportunities to individuals that have a statistically significant amount of impact on the health indicators of individuals rather than the recommendations with specific services opportunities that do not have a statistically significant impact on the health indicators of individuals.

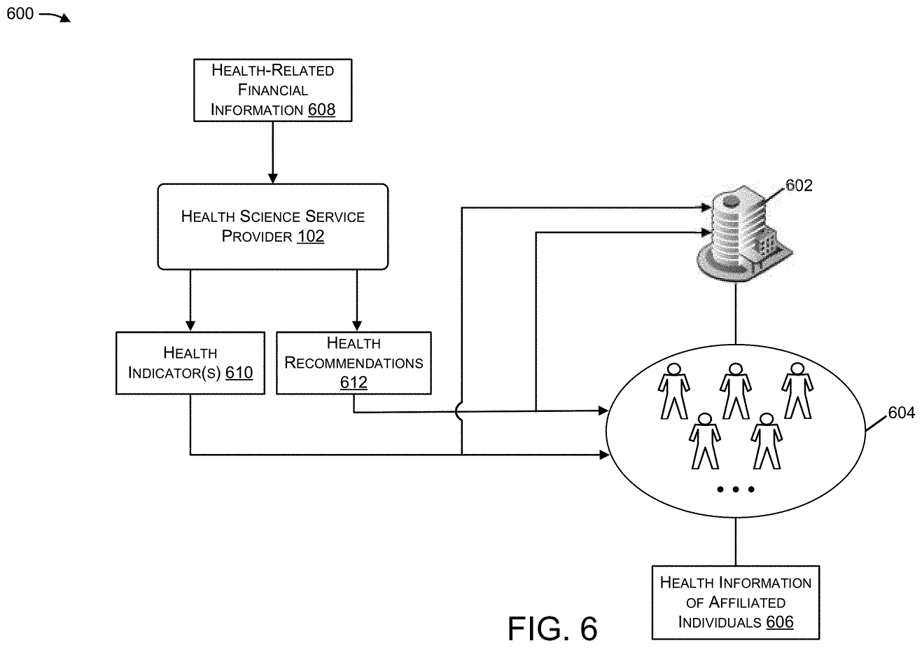

FIG. 6 illustrates an architecture 600 to utilize health information of individuals associated with an enterprise 602 to determine health indicators of the affiliated individuals 604 and to determine health recommendations with specific services opportunities for the affiliated individuals 604. The affiliated individual 604 can include individuals that are in some manner associated with the enterprise 602. For example, the affiliated individuals 604 can include employees of the enterprise 602. The affiliated individuals 604 can also include family members of employees of the enterprise 602. Additionally, the affiliated individuals 604 can include other individuals that work for the enterprise 602, such as contract workers that are not employees. In certain implementations, the affiliated individuals 604 can include individuals that obtain goods and/or services from the enterprise 602.

The affiliated individuals 604 can have health information 606. The health information 606 can be specific to the affiliated individuals 604 and can include at least one of genetic data, medical records, nutritional data, physical activity data, healthcare costs, wellness programs participation, behavioral data, or historical healthcare data (e.g., healthcare provider visits, healthcare procedures performed, diagnoses of disease, interventions prescribed, etc.). The enterprise 602 can store at least portions of the health information 606. Additionally, portions of the health information 606 can be stored in association with other entities, such as healthcare providers. The health information 606 can be accessible to the health science service provider 102.

In particular implementations, the health information 606 can be derived from additional information associated with the affiliated individuals 604. For example, the health science service provider 102 can determine that an affiliated individual 604 has a probability of having one or more biological conditions based at least partly on purchases of goods and/or services made by the affiliated individual 604, location information of the affiliated individual 604, media content consumed by the affiliated individual 604, electronic device usage by the affiliated individual 604, or combinations thereof.

The health science service provider 608 can also have access to health-related financial information 608. The health-related financial information 608 can include the financial costs associated with various aspects related to providing healthcare to individuals. In particular implementations, at least portions of the health-related financial information 608 can correspond to healthcare costs of the enterprise 602 with respect to the affiliated individuals 604. For example, the health-related financial information 608 can include premiums or portions of premiums paid by the enterprise 602 to provide health insurance for the affiliated individuals 604. In other examples, the health-related financial information 608 can include costs of wellness programs and/or other health-related benefits programs offered by the enterprise 602 on behalf of the affiliated individuals 604. The health-related financial information 608 can also include costs paid by one or more insurance companies associated with the enterprise 602 for healthcare to be provided to the affiliated individuals 604. Additionally, the health-related financial information 608 can include healthcare costs of enterprises other than the enterprise 602.

The health science service provider 102 can analyze at least portions of the health-related financial information 608 and at least portions of the health information 606 of the affiliated individuals 604 to determine health indictors 610 of the affiliated individuals 604 and health recommendations 612 for the affiliated individuals 604. The health indicators 610 can include health scores of the affiliated individuals 604 that correspond to a level of health of the affiliated individuals 604. In particular implementations, the health indicators 610 can indicate a 10-year mortality risk. The health indicators 610 can be generated by the health science service provider 102 based on one or more models generated by the health science service provider 102. In certain implementations, the health science service provider 102 can send the health indicators 610 to the affiliated individuals 604 and/or to the enterprise 602.

The health recommendations 612 can include recommendations with specific services opportunities for actions that the affiliated individuals 604 can take to impact their respective health indicators 610. For example, the health recommendations 612 can indicate physical activity that an affiliated individual 604 can take to increase one or more of their health indicators 610. The health recommendations 612 can also indicate nutritional content that can increase one or more health indicators 610 and/or medication that can increase one or more health indicators 610. The health recommendations with specific service opportunities 612 can also indicate an amount of impact that performing the health recommendations with specific services opportunities 612 can have on one or more health indicators 610 of an affiliated individual 604.

In particular implementations, the health recommendations with specific services opportunities 612 can be based on behavioral information associated with the affiliated individuals 604. For example, the health science service provider 102 can determine health recommendations with specific services opportunities 612 that an affiliated individual 604 is likely to act upon. In various implementations, the health science service provider 102 can determine health recommendations with specific services opportunities 612 based on interests of affiliated individuals 604, hobbies of affiliated individuals 604, habits of affiliated individuals 604, or combinations thereof. To illustrate, the health science service provider 102 can determine that a health recommendation with specific services opportunities 612 for an affiliated individual 604 that can impact a health indicator 610 of the affiliated individual 604 is riding a bicycle to a park with their child based on the affiliated individual 604 living within a mile of the park, having recently purchased a bicycle, and that physical activity is likely to impact the body mass index of the affiliated individual 604 in a positive way.

Additionally, the health recommendations with specific service opportunities 612 can be determined based on behavioral profiles of the affiliated individuals 604. The behavioral profiles can be determined by the health science service provider 102 based on results of one or more behavioral assessments taken by the affiliated individuals 604. In various implementations, the behavioral profiles of the affiliated individuals 604 can be based on actions taken by the affiliated individuals 604 via electronic devices of the affiliated individuals 604, such as interactions with one or more applications executed by the electronic devices of the affiliated individuals 604. Further, the behavioral profiles of the affiliated individuals 604 can be based on financial transactions of the affiliated individuals 604.

The behavioral profiles of the affiliated individuals 604 can be analyzed with respect to one or more of the health recommendations with specific service opportunities 612 to determine particular health recommendations with specific service opportunities 612 for certain ones of the affiliated individuals 604. In particular implementations, the health science service provider 102 can generate a vector for the affiliated individuals that includes a score for each affiliated individual 604 with respect to respective health recommendations with specific service opportunities 612. For a given individual, the score for each recommendation with specific servicer opportunities can be based on behavioral characteristics associated with the recommendation and the behavioral profile of the given individual. For example, the health science service provider 102 can determine a first score for a first affiliated individual for having an appointment with a genetic counselor, a second score for the first affiliated individual for downloading and utilizing a food tracking mobile device application, and a third score for the first affiliated individual for obtaining a membership to a fitness facility. Continuing with this example, the second score for the first affiliated individual can be greater than the first score and the third score and also be above a threshold level where the threshold level indicates a probability that the individual will follow through with the recommendation. In this situation, the health science service provider 102 can include a recommendation in the health recommendations with specific service opportunities 612 for the first affiliated individual to download and utilize a food tracking mobile device application to improve a health indicator 610 of the first affiliated individual. In additional examples, the health science service provider 102 can determine an additional first score, an additional second score, and an additional third score for a second affiliated individual where the additional first score, the additional second score, and the additional third score are different from the first score, the second score, and the third score of the first affiliated individual and the differences in the scores can be based on differences in the behavioral characteristics of the first affiliated individual and the second affiliated individual. In this scenario, a health recommendation with specific service opportunities 612 for the second affiliated individual can be for the second affiliated individual to visit a genetic counselor.

The health recommendations with specific services opportunities 612 can also be based on a financial cost of implementing the health recommendations with specific service opportunities 612. In some cases, the financial cost can be incurred by the enterprise 602, the affiliated individuals 604, or combinations thereof. In certain implementations, the health science service provider 102 can weigh the financial costs of various health recommendations with specific service opportunities 612 with the estimated benefit and provide the health recommendations with specific services opportunities 612 with an optimized cost to benefit ratio to the enterprise 602 and/or to the affiliated individuals 604.

In various implementations, the health service provider 102 can determine health recommendations with specific service opportunities 612 to the enterprise 602 that are effective in impacting the health indicators 610 of the affiliated individuals 604. For example, the health science service provider 102 can analyze the health information 606 to determine healthcare providers that are associated with positively impacting the health indicators 610 of the affiliated individuals 604. In other examples, the health science service provider 102 can analyze the health information 606 to determine particular interventions that have a positive impact on the health indicators 610 of the affiliated individuals 604. In an illustrative example, the health science service provider 102 can determine that a wellness program of the enterprise 602 where affiliated individuals 604 using certain physicians near the enterprise 602 during lunchtime provides positive impact to the health indicators 610 of the affiliated individuals 604 and the health science service provider 102 can prioritize these physician in the health recommendations with specific service opportunities 612 sent to individuals 604 on behalf of the enterprise 602.

The health science service provider 102 can also determine whether affiliated individuals 604 are receiving recommended health care with specific service opportunities for those affiliated individuals 604. To illustrate, the health science service provider 102 can determine a biological condition of an affiliated individual 604 and also determine recommended care with specific services opportunities for the biological condition. In certain implementations, the recommended care with specific services opportunities can be determined based on information included in medical literature, medical guidelines, or both. The health science service provider 102 can then analyze health-related financial information 608 and/or health information 606 for the affiliated individual 604 to determine an amount of the recommended care with specific services opportunities that the affiliated individual 604 has obtained and provide health recommendations with specific service opportunities 612 to the enterprise 602 indicated the amount of the recommended care that the affiliated individual 604 has received for the biological condition. The health recommendations with specific services opportunities 612 provided to the enterprise 602 can also indicate additional care that the affiliated individual 604 can obtain such that the affiliated individual 604 can have an increased amount of the recommended care for the biological condition.

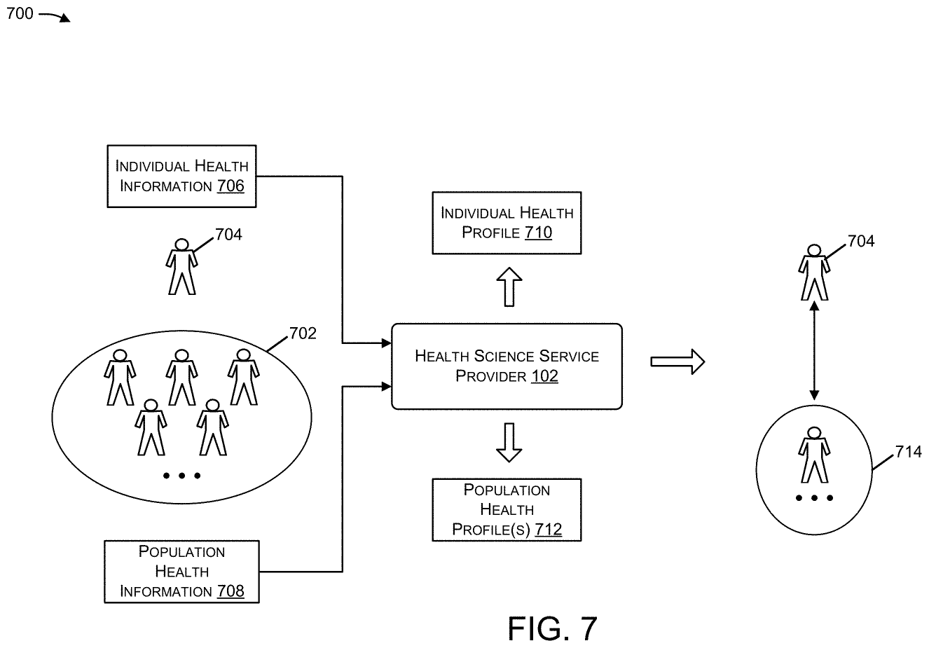

FIG. 7 illustrates an architecture 700 to identify one or more individuals within a population 702 that represent another individual 704 in a different state of health and/or at a different life stage. In particular implementations, the health science service provider 102 can obtain de-identified individual health information 706 and population health information 708. The individual health information 706 can include various types of health-related information of the individual 704, such as medical records of the individual 704, wearable device data of the individual 704, healthcare history of the individual 704, physical characteristics of the individual 704 (e.g., height, weight, etc.), biological characteristics of the individual 704 (e.g., cholesterol levels, blood pressure levels, medical imaging, microbiome, environmental etc.), genetic characteristics of the individual 704, combinations thereof, and so forth. Additionally, the population health information 708 can include similar information for the individuals included in the population 702.

The health science service provider 102 can analyze the individual health information 706 to determine an individual health profile 710 for the individual 704. The health science service provider 102 can also analyze the population health information 708 to generate population health profiles 712 for the individuals included in the population 702. The health profiles 710, 712 can indicate various health-related characteristics and can include certain genetic characteristics, certain physical features, certain biological features, certain healthcare history features, certain nutritional habits, certain physical activity habits, combinations thereof, and so forth. For example, the individual health profile 710 can indicate that the individual 704 has an age of 28, a height of 5 feet and ten inches, a weight of 172 pounds, a genetic marker for Alzheimer's disease, and has participates in moderate exercise 3 times per week on average. The health profiles 710 can also include health indicators, such as health scores, of the individual 704 and the individuals included in the population 702.

The health science service provider 102 can utilize the individual health profile 710 to identify one or more individuals 714 included in the population that had a similar health profile as the individual 704 in the past. That is, the health science service provider 102 can identify an individual included in the population that is currently 38 years old and had a health profile that has at least a threshold similarity with the individual health profile 710 ten years ago. In some situations, the one or more individuals 714 can be considered “digital twins” of the individual 704. The health science service provider 102 can utilize the current health profile of the one or more individuals 714 and their health profiles over time to determine the impact of certain interventions on the health indicators of the individual 704. For example, the health profiles of the one or more individuals 714 can indicate that these individuals had an intake of 6 servings of fruits and vegetables daily and had at least 7 hours of sleep on average from age 28 to age 38. Continuing with this example, the health science service provider 102 can determine an impact on the health indicators of the individual 704 of having an intake of 6 servings of fruits and vegetables daily and had at least 7 hours of sleep on average based on the health indicators of the one or more individuals 714 over the past 10 years. In this way, the health science service provider 102 can improve the accuracy of the health recommendations with specific service opportunities made to the individual 704.

In illustrative implementations, the health profiles of individuals can be characterized as vectors that include components corresponding to respective risk factors of one or more biological conditions. The health science service provider 102 can compare the vectors of individuals to determine cohorts of individuals that can be utilized to determine health recommendations. In particular examples, the health science service provider 102 can determine Euclidean distances between the health profile vectors of individuals and determine a group of individuals that have less than a threshold Euclidean distance between their health profiles. In this way, the health science service provider 102 can determine a cohort of individuals 714 that corresponds to the individual 704.

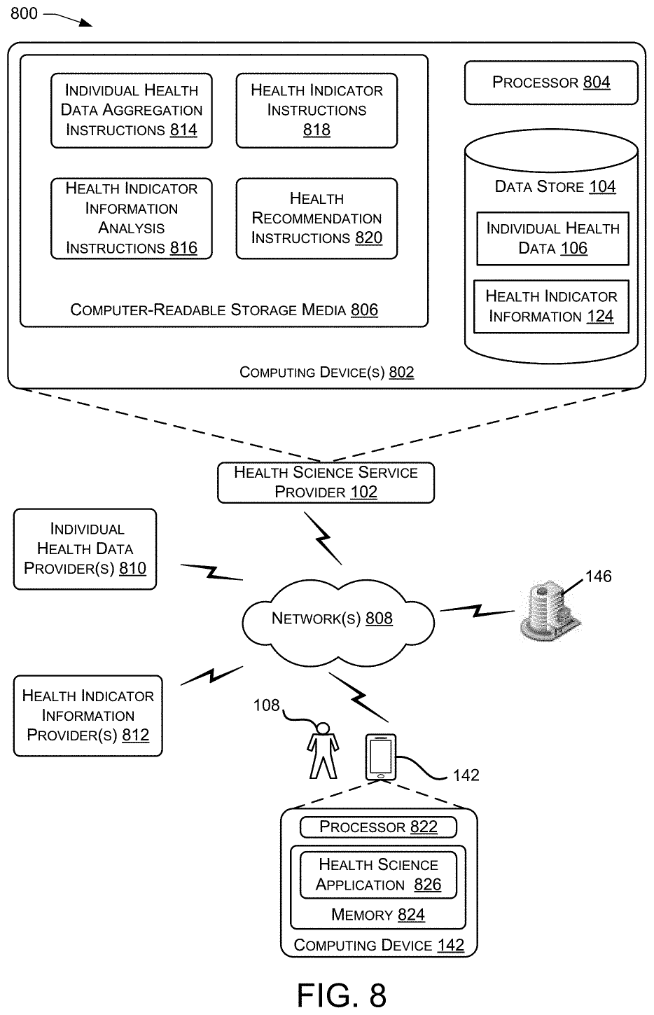

FIG. 8 is a block diagram illustrating an example system 800 to determine indicators of the health of individuals. The system 800 can include one or more computing devices 802. The one or more computing devices 802 can be associated with the health science service provider 102. In implementations, the one or more computing devices 802 can be included in a cloud computing architecture that operates the one or more computing devices 802 on behalf of the health science service provider 102. In these scenarios, the cloud computing architecture can implement one or more virtual machine instances on behalf of the health science service provider 102 on the one or more computing devices 802. The cloud computing architecture can be located remotely from the health science service provider 102. In additional implementations, the one or more computing devices 802 can be under the direct control of the health science service provider 102. For example, the health science service provider 102 can maintain the one or more computing devices 802 to perform operations related to determining indicators of the health of individuals. In various implementations, the one or more computing devices 802 can include one or more server computers.

The one or more computing devices 802 can include one or more processors, such as processor 804. The one or more processors 804 can include at least one hardware processor, such as a microprocessor. In various implementations, the one or more processors 804 can include a central processing unit (CPU), a graphics processing unit (GPU), or both a CPU and GPU, or other processing units. Additionally, the one or more processors 804 can include a local memory that may store program modules, program data, and/or one or more operating systems.

In addition, the one or more computing devices 802 can include one or more computer-readable storage media, such as computer-readable storage media 806. The computer-readable storage media 806 can include volatile and nonvolatile memory and/or removable and non-removable media implemented in any type of technology for storage of information, such as computer-readable instructions, data structures, program modules, or other data. Such computer-readable storage media 806 can include, but is not limited to, RAM, ROM, EEPROM, flash memory or other memory technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, removable storage media, or any other medium that can be used to store the desired information and that can be accessed by a computing device. Depending on the configuration of the one or more computing devices 802, the computer-readable storage media 806 can be a type of tangible computer-readable storage media and can be a non-transitory storage media.

The one or more computing devices 802 can include one or network interfaces (not shown) to communicate with other computing devices via one or more networks 808. The one or more networks 808 can include one or more of the Internet, a cable network, a satellite network, a wide area wireless communication network, a wired local area network, a wireless local area network, or a public switched telephone network (PSTN). In various implementations, the one or more computing devices 802 can exchange information with one or more individual health data providers 810 via the one or more networks 808. The one or more individual health data providers 810 can obtain and/or store health information for a number of individuals. The one or more individual health data providers 810 can include entities that obtain and/or store individual health data, computing devices that obtain and/or store individual health data, or combinations thereof. Additionally, the one or more computing devices 802 can exchange information with one or more health indicator information providers 812 via the one or more networks 808. The health indicator information providers 812 can, in particular implementations, include clinical trials information providers, medical research information providers, health model development platforms, or combinations thereof. Further, the one or more computing devices 802 can exchange information with the enterprise 146.

The computer-readable storage media 806 can be used to store any number of functional components that are executable by the one or more processors 804. In many implementations, these functional components comprise instructions or programs that are executable by the one or more processors 804 and that, when executed, implement operational logic for performing the operations attributed to the one or more computing devices 802. Functional components of the one or more computing devices 802 that can be executed on the one or more processors 804 for implementing the various functions and features related to determining indicators of individual health, as described herein, include individual health data aggregation instructions 814, health information analysis instructions 816, health indicator instructions 818, and health recommendation instructions 820.

Additionally, the one or more computing devices 802 can include one or more input/output devices (not shown). The one or more input/output devices can include a display device, keyboard, a remote controller, a mouse, a printer, audio input/output devices, a speaker, a microphone, a camera, and so forth

The one or more computing devices 802 can also include, or be coupled to, the data store 104 that can include, but is not limited to, RAM, ROM, EEPROM, flash memory, one or more hard disks, solid state drives, optical memory (e.g. CD, DVD), or other non-transient memory technologies. The data store 104 can maintain information that is utilized by the one or more computing devices 802 to perform operations related to determining indicators of the health of individuals. For example, the data store 104 can store the individual health data 106 and the health indicator information 124.

The individual health data aggregation instructions 814 can be executable by the one or more processors 804 to obtain the individual health data 106 and store the individual health data 106 in the data store 104. The individual health data aggregation instructions 814 can query various databases to obtain portions of the individual health data 106. For example, the individual health data aggregation instructions 814 can cause communications to take place between the one or more computing devices 802 and the one or more individual health data providers 810 to obtain portions of the individual health data 106, such as medical records of the individuals, laboratory test results of individuals, medical imaging data, physical activity information of individuals, nutritional information of individuals, genetic information of individuals, behavioral, environmental data other health related information of individuals, or combinations thereof. In particular implementations, the individual health data aggregation instructions 814 can obtain digital authorization to access health information of individuals stored by the one or more individual health data providers 810. The individual health data aggregation instructions 814 can also cause communications to take place between the one or more computing devices 802 and computing devices of individuals to obtain portions of the individual health data 106. In an illustrative example, the individual health data aggregation instructions 814 can cause communication to take place with the computing device 142 of the individual 108 to obtain physical activity data of the individual 108, physiological measurements of the individual 108, such as pulse rate and/or blood pressure, or both.

Further, the individual health data aggregation instructions 814 can store the portions of the individual health information 106 in the data store 104 in accordance with particular data structures and/or in accordance with particular formats to store data. In illustrative implementations, upon obtaining health data of the individual 108, the individual health data aggregation instructions 814 can determine the type of health data that was obtained and identify a storage location for the health data. For example, the individual health data aggregation instructions 814 can obtain blood pressure data of the individual 108 and determine storage locations associated with the individual 108 in the data store 104 and also determine particular storage locations for blood pressure data of the individual 108 within the data store 104. In various implementations, the individual health data aggregation instructions 814 can also retrieve portions of the individual health data 106 upon request. In various implementations, the individual health data 106 can include behavioral profiles of individuals.

The health information analysis instructions 816 can be executable by the one or more processors 804 to analyze the health indicator information 124 to determine factors that can impact the health of individuals. In various implementations, the health information analysis instructions 816 can parse the health indicator information 124 to identify models that can be utilized to determine indicators of the health of individuals. In certain implementations, the health information analysis instructions 816 can combine at least portions of a plurality of models included in the health indicator information 124. Additionally, the health information analysis instructions 816 can generate new models to determine the health of individuals. For example, the health information analysis instructions 816 can identify various factors that can impact the health of individuals from the health indicator information 124. The factors may or may not be a part of a pre-existing model included in the health indicator information 124. The health information analysis instructions 816 can then combine the factors into a particular model that can be utilized to determine indicators of the health of individuals.

The factors determined by the health information analysis instructions 816 that can be utilized to determine indicators of the health of individuals can be based on characteristics of certain populations. For example, some factors that can impact the health of a first population of individuals may not have an impact or may not have as great of an impact on the health of individuals included in a different population. Additionally, the factors determined by the health information analysis instructions 816 to determine indicators of the health of individuals can be specific to particular individuals. In particular implementations, the factors that can impact the health of individuals can be based on characteristics of individuals, such as physical characteristics, genetic characteristics, physiological characteristics, medical imaging, lifestyle characteristics, behavior, environmental data or combinations thereof. The physical characteristics of individuals can include age, height, weight, and the like, while the genetic characteristics of individuals can include the presence or absence of genes that can impact the health of individuals. The physiological characteristics of the individuals can include thyroid hormone levels, cholesterol levels, and the amount of other biological molecules present in the bodies of individuals and the lifestyle characteristics of individuals can include amount of alcohol consumption, amount of physical activity, nutrient intake, and the like.

The health indicator instructions 818 can be executable by the one or more processors 804 to determine health indicators for individuals, such as health scores. The health indicators can represent a level of health for a particular individual. In various implementations, multiple health indicators can be determined for each individual. In additional implementations, the health indicators can comprise various components related to different aspects of the health of individuals. For example, the health indicator instructions 818 can determine an overall health indicator for individuals and sub-indicators related to different components of health, such as a health score for cardiac health, a health score for physical activity, a health score for nutritional intake, a health score for diabetes related health, and so forth.

The health indicator instructions 818 can determine health indicators for individuals by evaluating factors identified as impacting the health of individuals in relation to the health data of the individuals. In particular implementations, the health indicator instructions 818 can identify or obtain one or more models that include a number of factors that can impact the health of individuals and implement the one or more models with respect to the health data of the individuals to generate one or more health indicators. In implementations, a model applied to generate a health score for a first group of individuals can be different from another model applied to generate a health score for a second group of individuals.

The health recommendation instructions 820 can be executable by the one or more processors 804 to generate recommendations to optimize the health of individuals. The recommendations can indicate actions for individuals to take to increase their health indicators. In certain implementations, health recommendation instructions 820 can utilize the factors that can impact the health of an individual and determine actions that can modify the impact of the factors to increase the health indicators for the individual. The health recommendation with specific services opportunities instructions 820 can also modify the values of an individual for a number of health factors that can impact the health of the individual. For example, in situations where blood glucose level is a factor that can affect the health of an individual, the health recommendation with specific services opportunities instructions 820 can generate a recommendation to reduce a percentage of daily caloric intake attributable to simple sugars to a certain percentage to minimize any detrimental impact on the health of the individual based on an elevated blood glucose level for the individual. In situations where an individual's blood glucose level is lower than a threshold level, the health recommendation with specific services opportunities instructions 820 can determine actions that can increase the blood glucose level of the individual, such as eating a number of servings of fruit daily.

In various implementations, as the health indicators for an individual change over time, the health recommendation with specific services opportunities instructions 820 can generate updated, modified, or new recommendations with specific services opportunities to increase the health indicators for the individual. In illustrative implementations, the individual health data 106 for an individual can change over time and affect the health indicators generated for the individual. For example, as the blood pressure of an individual decreases, yet cholesterol levels are at a relatively high, the health recommendation with specific services opportunities instructions 820 can generate recommendations related to modifying nutritional habits of the individual whereas previous recommendations may have been related to modifying physical activity and nutritional habits.

The health recommendation with specific service opportunities instructions 820 can also identify health recommendations with specific service opportunities that impact the health indicators of individuals. For example, the health recommendation with specific services opportunities instructions 820 can analyze changes in health indicators of individuals, health recommendations with specific services opportunities made to individuals, and health recommendations with specific services opportunities implemented by individuals. The health recommendation with specific service opportunities instructions 820 can then determine health recommendations that, when implemented by individuals, have a statistically significant impact on the health indicators of the individuals. In certain implementations, the health recommendations with service opportunities that have an impact on the health indicators of individuals can be based on behavioral profiles of the individuals with respect to the respective recommendations. In illustrative examples, the health recommendation with specific services opportunities instructions 820 can implement one or more neural networks to determine the recommendations with service opportunities that have an impact on the health indicators of individuals. Upon identifying health recommendations with specific services opportunities that have an impact on the health indicators of various individuals, the health recommendation instructions with specific service opportunities 820 can make recommendations to individuals that are tailored to their health profile and to their behavioral profile that are more likely than other health recommendations to result in an improvement to the health indicators of the individuals.

The computing device 142 can include a mobile phone, a smart phone, a tablet computing device, a laptop computing device, a portable gaming device, a desktop computing device, combinations thereof, and the like. The computing device 142 can include one or more processors, such as processor 822 and memory 824. The one or more processors 822 can include at least one hardware processor, such as a microprocessor. In some cases, the one or more processors 822 may include a central processing unit (CPU), a graphics processing unit (GPU), or both a CPU and GPU, or other processing units. Additionally, the one or more processors 822 can include a local memory that may store program modules, program data, and/or one or more operating systems.

The memory 824 can include volatile and nonvolatile memory and/or removable and non-removable media implemented in any type of technology for storage of information, such as computer-readable instructions, data structures, program modules, or other data. The computing device 142 can also include one or network interfaces (not shown) to communicate with other computing devices via the one or more networks 808. Additionally, the computing device 142 can include one or more input/output devices (not shown).

The memory 824 can be used to store any number of functional components that are executable by the one or more processors 822. In many implementations, these functional components comprise instructions or programs that are executable by the one or more processors 822 and that, when executed, implement operational logic for performing the operations attributed to the computing device 142. Functional components of the computing device 142 that can be executed on the one or more processors 822 for implementing the various functions and features related to health indicators and health recommendations for individuals, as described herein, can include a health science application 826. The health science application 826 can provide one or more user interfaces to provide information regarding health indicators for the individual 108 and information related to recommendations with specific services opportunities to increase the health indicators of the individual 108. In various implementations, the health science app 826 can obtain portions of the health data of the individual 108 and send the health data of the individual to the health science service provider 102.

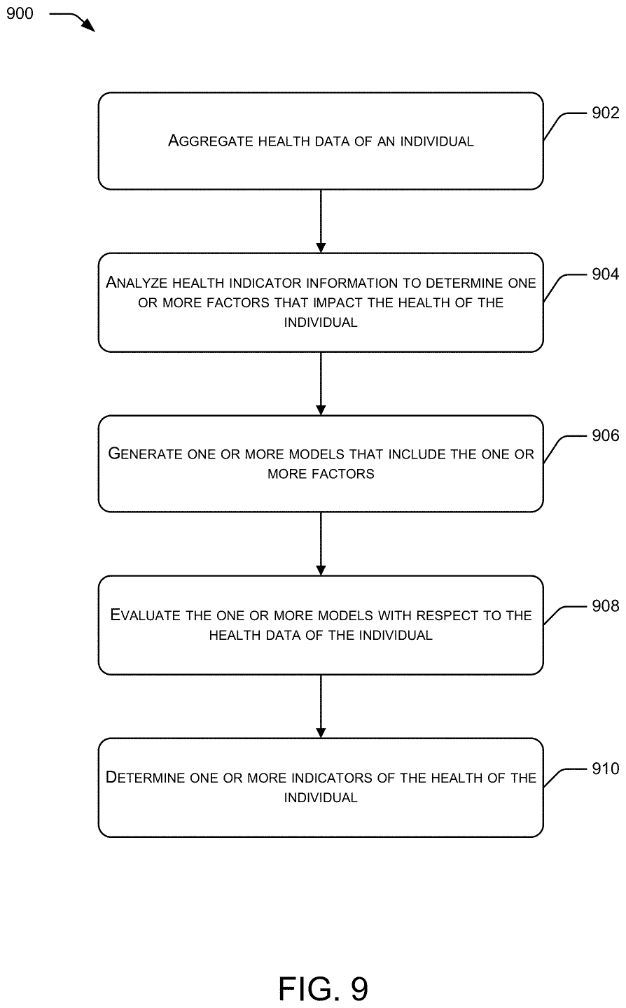

FIGS. 9-12 illustrate example processes related to providing health indicators and health recommendations with specific service opportunities to individuals. These processes (as well as each process described herein) are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the process.

FIG. 9 is a flow diagram of an example process 900 to determine indicators of the health of individuals. At 902, the process 900 includes aggregating health data of an individual. The health data of the individual can be obtained from computing device of the individual. In certain implementations, the computing device of the individual can be executing applications that are collecting and storing data related to the health of the individual. In particular implementations, the computing device can include and/or be coupled to sensors that generate physiological data of the individual. Aggregating the health data of the individual can also include accessing medical records of the individual and laboratory tests of the individual. In various implementations, the individual can provide the medical records, laboratory test results and medical imaging among other data. In additional implementations, the medical records, laboratory test and medical imaging data can be obtained from entities storing this information, such as doctors' offices, hospitals, and other healthcare providers and vendors.

At 904, the process 900 includes analyzing health indicator information to determine one or more factors that impact the health of the individual. The health indicator information can include medical research, clinical trials information and other sources of data. The health indicator information can be analyzed by identifying factors shown by the medical research and/or the clinical trials to have a positive or negative impact on the health of the individuals. Analyzing the health indicator information can include matching characteristics of the individuals included in the medical research and/or clinical trials with characteristics of the individual for which the health indicator is being generated. The factors that impact the health of the population that participated in the medical research and/or clinical trials that most closely correspond to the individual can be the factors identified by operation 904.

At 906, the process 900 includes generating one or more models that include the one or more factors identified in operation 904. In particular implementations, the one or more factors can be included in a single model included in the health indicator information. In additional implementations, the one or more factors can be identified with respect to multiple medical research studies, multiple clinical trials or other sources of data. In these situations, the factors from these studies, trials and other sources of data can be combined into a single model. The one or more models can also include at least one weighting for each factor that indicates an amount of impact that the factor has on the health of the individual. In certain implementations, the one or more models can apply to a relatively large population, while in other implementations, the one or more models can apply to a relatively small population. In illustrative implementations, the one or more models can be personalized with respect to the individual.

At 908, the process 900 includes evaluating the one or more models with respect to the health data of the individual. To illustrate, for factors included in the one or more models, the health data of the individual that correspond to those factors can be obtained. For example, in a model where cholesterol levels are a factor, the cholesterol levels of the individual can be obtained. The health data of the individual related to the factors can be quantified to produce a value for the individual with respect to the factor. Any weightings associated with the factors can also be applied to the value as part of the evaluation of the one or more models in relation to the health data of the individual.

At 910, the process 900 includes determining one or more indicators of the health of the individual. The one or more indicators can include one or more health scores for the individual. The one or more health indicators can be determined by generating values for each of the factors included in the one or more models and producing a sum of the values for the factors. In an illustrative example, the one or more health indicators can be determined by producing a first value for an individual with respect to nutritional intake, a second value for the individual with respect to blood pressure, and a third value for the individual with respect to an amount of physical activity of the individual. A health indicator for the individual can be determined by adding the first value, the second value, and the third value. In additional implementations, multiple health indicators can be determined for the individual. To illustrate, a nutritional intake health indicator can be determined for the individual in addition to a blood pressure health indicator and a physical activity health indicator.

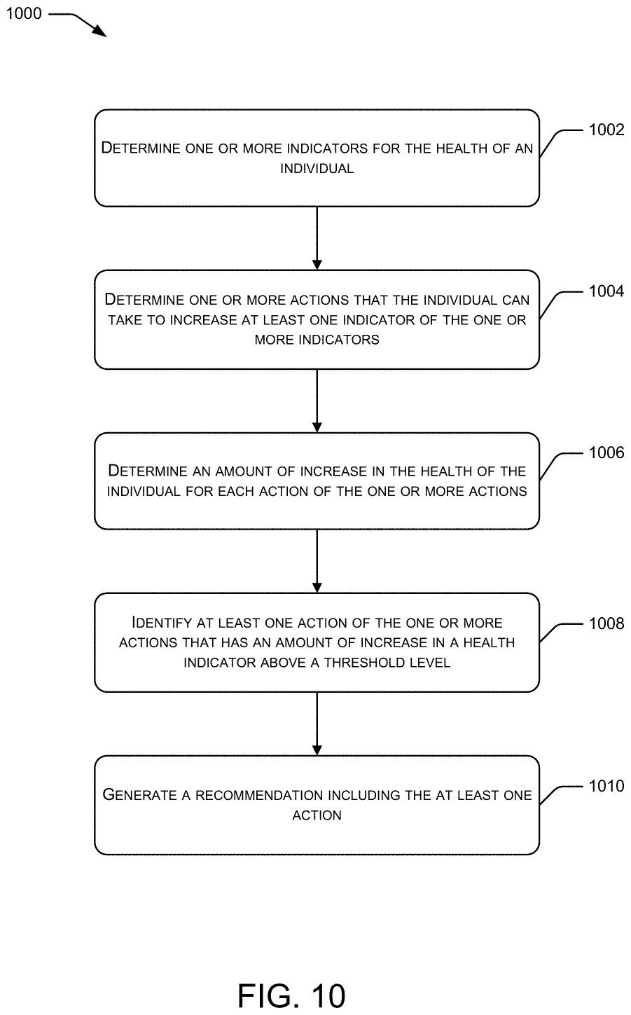

FIG. 10 is a flow diagram of an example process 1000 to determine recommendations to improve the health scores of individuals. At 1002, the process 1000 includes determining one or more indicators for the health of the individual. In particular implementations, the one or more indicators of the health of the individual can be determined according to the process 900 described with respect to FIG. 9.

At 1004, the process 1000 includes determining one or more actions that the individual can take to increase at least one indicator of the one or more indicators. In various implementations, a health score of an individual can be included in a scale of scores. In implementations, particular ranges of scores can be associated with corresponding actions that can be implemented to improve the health score of an individual. In additional implementations, actions that an individual can take to increase their health score can be based on certain factors that were utilized to determine the health score of the individual. In an illustrative example, going for a jog or taking a 1-mile walk can be actions associated with increasing a health score by increasing a physical activity factor, while eating fruits and vegetables can be associated with increasing a health score by increasing a nutritional intake factor.

At 1006, the process 1000 includes determining an amount of improvement in the health of the individual for each action of the one or more actions. In particular implementations, an amount of increase in a health score that can be generated by taking a particular action can be based at least partly on an amount of impact that the action can have on the health of the individual. In an illustrative example, increasing the amount of nuts consumed by an individual can have an appreciable of impact on cardiac health of an individual, while increasing the amount of nuts consumed by an individual has less impact on the blood glucose level of the individual. Additionally, an amount of increase in the health score associated with taking an action can be based at least partly on a value for a factor that can be increased by taking the action. For example, an individual having a relatively high value for a physical activity factor would not necessarily receive a corresponding amount of increase in their health score by taking a mile long walk each day.

At 1008, the process 1000 includes identifying at least one action of the one or more actions that has an amount of improvement above a threshold level. In implementations, the threshold level can be set such that at least a minimum amount of impact to the health score of the individual is predicted to occur based on taking the action. At 1010, the process 1000 includes generating a recommendation including the at least one action. For example, an action having a threshold amount of increase of the health score of the individual can be included in an email or text message sent to a computing device of the individual. Additionally, an action having a threshold amount of increase of the health score of the individual can be accessible via a health science application executing on a computing device of the individual and/or via a website accessible by the individual.

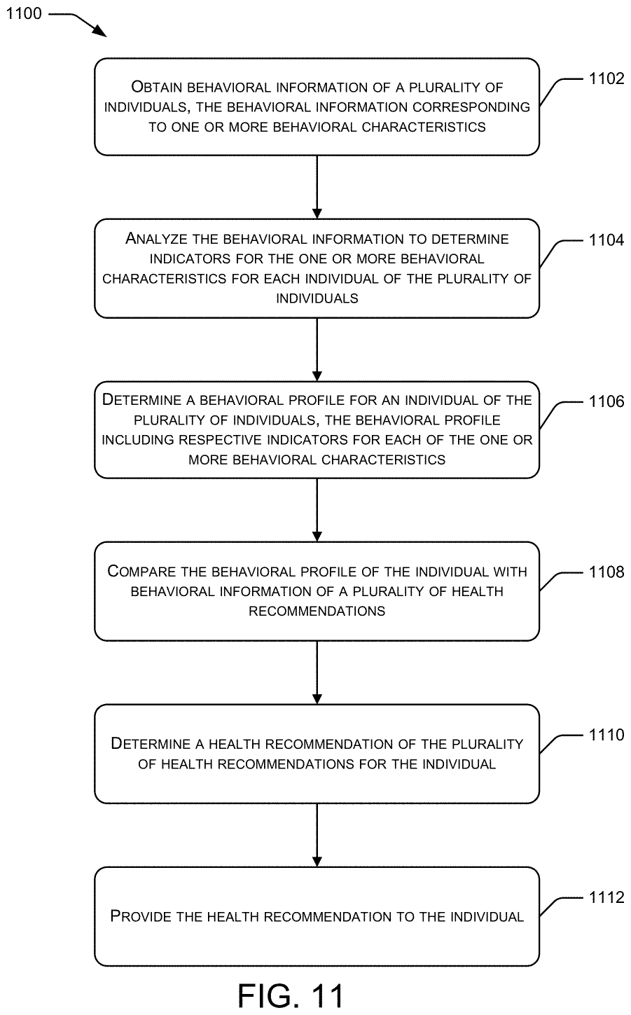

FIG. 11 is a flow diagram of an example process 1100 to determine recommendations to improve the health scores of individuals based on behavioral characteristics of the individuals. The process 1100 can include, at 1102, obtaining behavioral information of a plurality of individuals. The behavioral information can correspond to one or more behavioral characteristics. In particular implementations, the behavioral information can be obtained from one or more behavioral evaluations performed with respect to the plurality of individuals. In additional implementations, the behavioral information can be determined based on at least one of respective location information for the plurality of individuals, financial transaction information for the plurality of individuals, or usage of one or more mobile device applications. Additionally, the one or more behavioral characteristics can include at least one of extraverted, introverted, sensing, intuition, thinking, feeling, judging, perception, honesty-humility, emotionality, extraversion, agreeableness, conscientiousness, openness to experience.

In addition, the process 1100 can, at 1104, include analyzing the behavioral information to determine indicators for the one or more behavioral characteristics for each individual of the plurality of individuals. In various implementations, determining the indicators for the one or more behavioral characteristics for each individual of the plurality of individuals can include determining a first numerical score along a scale for a behavioral characteristic of the one or more behavioral characteristics with respect to the individual. The first numerical score can be based at least partly on at least one of answers provided by the individual to questions included in a behavioral assessment, usage by the individual of one or more mobile device applications, financial transaction information related to the individual, or location information of the individual.

At 1106, the process 1100 can include determining a behavioral profile for an individual of the plurality of individuals. The behavioral profile can include respective indicators for each of the one or more behavioral characteristics. The indicators can be numerical scores that provide an indication of a magnitude of a behavioral characteristic with respect to a particular individual.

Further, at 1108, the process 110 can include comparing the behavioral profile of the individual with behavioral information of a plurality of health recommendations. In certain implementations, each recommendation with specific service opportunities of the plurality of health recommendations with specific service opportunities can be associated with a second numerical score for each behavioral characteristics of the plurality of behavioral characteristics. Thus, in a manner similar to the numerical scores associated with the behavioral characteristics for individuals, the health recommendations with specific service opportunities can each be associated with certain behavioral characteristics more closely than other health behavioral characteristics. That is, a first health recommendation with specific service opportunities may be more likely to be implemented by extraverted individuals that enjoy new experiences, while a second health recommendation with specific services opportunities can be more likely to be implemented by introverted individuals that are more emotionally sensitive and less open to new experiences.

At 1110, the process 1100 can include determining a health recommendation of the plurality of health recommendations with specific service opportunities for the individual. In particular implementations, determining the health recommendation with specific services opportunities of the plurality of health recommendations for the individual can include comparing numerical scores for the behavioral characteristics associated with each respective recommendation with specific services opportunities with the numerical scores for the behavioral characteristics of the individual. Further, the health recommendation with specific services opportunities can be determined by also determining that a difference between the first numerical scores and the second numerical scores is less than a threshold difference.

Additionally, at 1112, the process 1100 can include providing the health recommendation with specific service opportunities to the individual. In additional scenarios, health recommendations with specific services opportunities can be provided to an enterprise associated with the individual. A health science service provider can determine an additional health recommendation with specific services opportunities for the enterprise. The additional health recommendation with specific service opportunities can correspond to an effectiveness metric. The effectiveness metric can be based on at least one of providers visited by individuals affiliated with the enterprise, medical interventions implemented by the individuals affiliated with the enterprise, medical interventions prescribed to the individuals affiliated with the enterprise, or programs provided by the enterprise to individuals affiliated with the enterprise. Further, the effectiveness metric can be determined at least partly based on improvements in health indicators of the individuals affiliated with the enterprise.

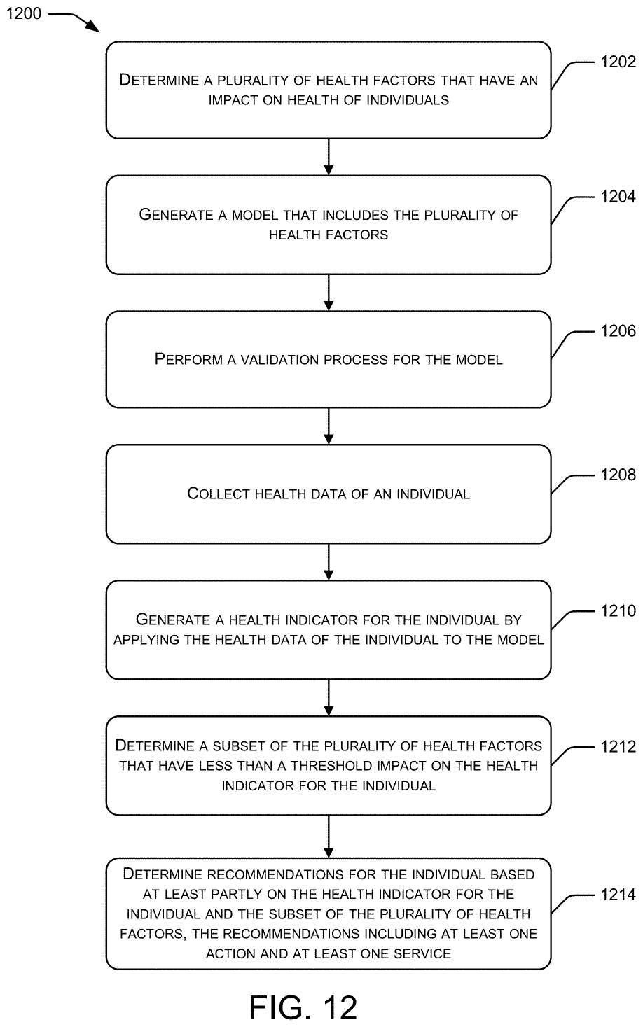

FIG. 12 is a flow diagram of an example process 1200 to utilize a model to determine a health indicator for an individual and recommendations for the individual to improve the health indicator. At 1202, the process 1200 includes determining a plurality of health factors that have an impact on health of individuals. The health factors may be determined by a health science service provider analyzing data that indicates biological conditions and characteristics of individuals that have the biological conditions. In certain implementations, at least a portion of the health factors can be identified in a medical literature database. In additional implementations, at least a portion of the health factors can be identified in a medical research study and/or a medical research paper.

At 1204, the process 1200 includes generating a model that includes the plurality of health factors. The model can include one or more coefficients that correspond to the plurality of health factors. In particular implementations, the model can include one or more additional factors that take into account characteristics of individuals whose data can be applied to the model. For example, the model can be adjusted for individuals based on the age of the individual and/or the sex of the individual.

At 1206, the process 1200 includes performing a validation process for the model. The validation process can be performed using a dataset that is different from a dataset used to determine the plurality of health factors that are included in the model. In various implementations, the plurality of health factors can be modified in situations where the model does not perform according to one or more performance metrics for the model. Additional health factors can be added to the model and one or more of the plurality of health factors can be removed from the model to improve the performance of the model during the validation process until the performance metrics of the model are within a specified tolerance level.

At 1208, the process 1200 includes collecting health data of an individual. The health data can include data obtained by sensors that can measure physiological activity and/or physiological characteristics of individuals. Additionally, the health data of the individual can include genetic information 112. The health data of the individual can also include activity data 114 for individuals. In certain implementations, the activity data 114 can indicate types of physical activity in which individuals can participate. In additional implementations, the health data can include medical imaging and laboratory test data 116 and/or nutrition information that can indicate types of food and beverages consumed by individuals.

At 1210, the process 1200 includes generating a health indicator for the individual by applying the health data of the individual to the model. The health indicator can correspond to a score that indicates a level of health of the individual. In particular implementations, the health indicator can correspond to a 10-year mortality risk for the individual. In various implementations, the 10-year mortality risk can be determined by evaluating the health data of the individual with respect to a plurality of possible causes of death for the individual.

At 1212, the process 1200 includes determining a subset of the plurality of health factors that have less than a threshold impact on the health indicator of the individual. In illustrative examples, the health science service provider can determine health factors that make the least amount of impact on increasing the health indicator of the individual. That is, the health science service provider can determine one or more health factors that weigh down the health indicator of the individual. The health science service provider can also determine a different subset of the health factors of the individual that have the greatest contribution to the health indicator of the individual. In some examples, the health science service provider can one or more health factors that most positively impact the health indicator of the individual.

At 1214, the process 1200 includes determining recommendations for the individual based at least partly on the health indicator for the individual and the subset of the plurality of health factors. The recommendations can include at least one action and at least one service. For example, the at least one action can include an action the individual can take to help improve the health indicator of the individual. In an illustrative example, the subset of the plurality of health factors can include blood glucose level and the recommendations can include an action to eat 5 or more servings of fruits and vegetables per day. Additionally, a service that corresponds to improving the blood glucose levels of the individual can correspond to wearing a continuous blood glucose monitor. In certain implementations, the recommendations can include an action and/or a service that corresponds to the different subset of health factors for the individual to help the individual continue to have a good performance with respect to the health factors that are most positively impacting the health indicator of the individual.

In various implementations, health data of the individual can be collected continuously, and the health indicator can be determined at various times or intervals. In certain implementations, the subset of health factors having a positive impact and an additional subset of health factors having a negative impact can change over time. Additionally, the recommendations provided to the individual can also change over time as the health indicator changes over time and/or as the health factors having the greatest positive and/or negative impact on the health indicator of the individual change over time.

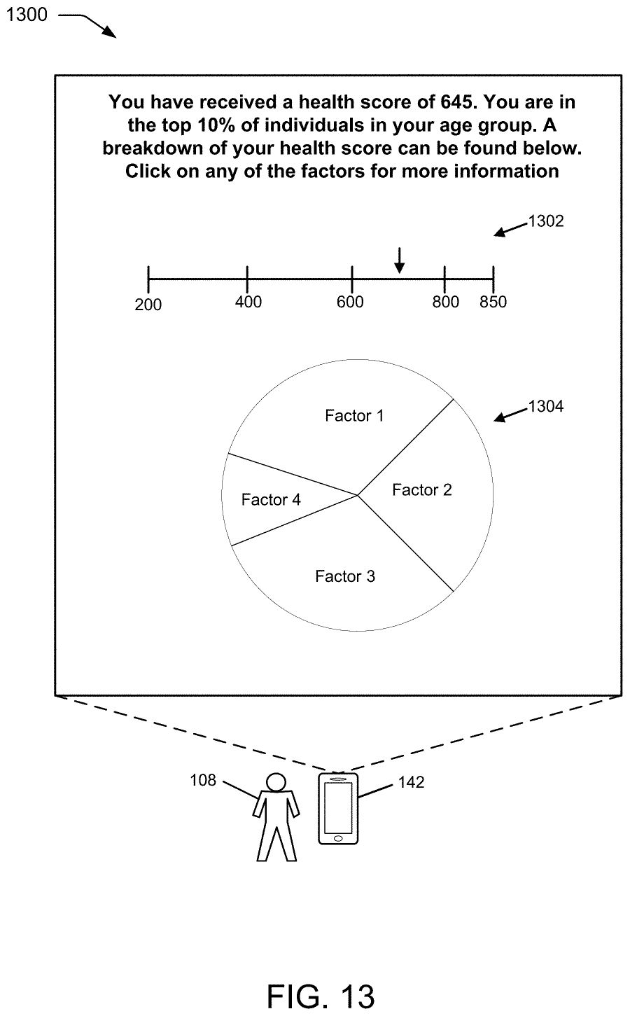

FIG. 13 is an example user interface 1300 including an indicator of the health of an individual. The user interface 1300 can be rendered and displayed on the computing device 142. The computing device 142 can be operated by the individual 108. The computing device 142 can include a mobile computing device, a smart phone, a tablet computing device, a laptop computing device, a desktop computing device, a gaming console, a portable gaming device, a multimedia device, combinations thereof, and the like. The user interface 1300 can be displayed in conjunction with a mobile device app executed by the computing device 142 or a website accessed by the computing device 142. In the illustrative example of FIG. 12, the user interface 1200 can be provided by a health science service provider, such as the health science service provider 102, and the user interface 1300 can include user interface elements directed to providing indications of the health of the individual 108.

The user interface 1300 indicates a health score of the individual and includes a scale 1302 that indicates a relative position of the score of the individual with respect to a range of scores that can be generated for individuals. The user interface 1300 indicates that a health score of the individual 108 is represented by the number 645 and that the health score is in the top 10% of health scores of individuals within a same age group as the individual 108. The user interface 1300 also includes a graphic 1304 that indicates four factors that were evaluated to determine the health score of the individual 108. The graphic 1304 also indicates the relative weightings that each of the factors was given in generating the health score of the individual 108 based on the area of the graphic 1304 attributed to the individual factors. The graphic 1304 indicates that some factors, such as Factor 1 and Factor 3, were weighted more heavily than Factor 2 and Factor 4 in determining the health score of the individual 108. In various implementations, the relative weightings of the factors included in the graphic 1304 can be different for additional individuals. For example, individuals included in a different age group can have different weightings for the factors utilized to generate the health score for the individual 108. In additional implementations, different factors can be utilized to determine the health score for other individuals. In an illustrative example, the first factor can correspond to activity level of the individual, the second factor can correspond to cholesterol levels of the individual 108, the third factor can correspond to blood glucose levels of the individual 108, and the fourth factor can correspond to tobacco use by the individual 108. In situations, where health scores are generated for other individuals having different characteristics than the individual 108, an additional factor, such as blood pressure or genetics, can be evaluated, and/or a factor can be removed, such as tobacco use.

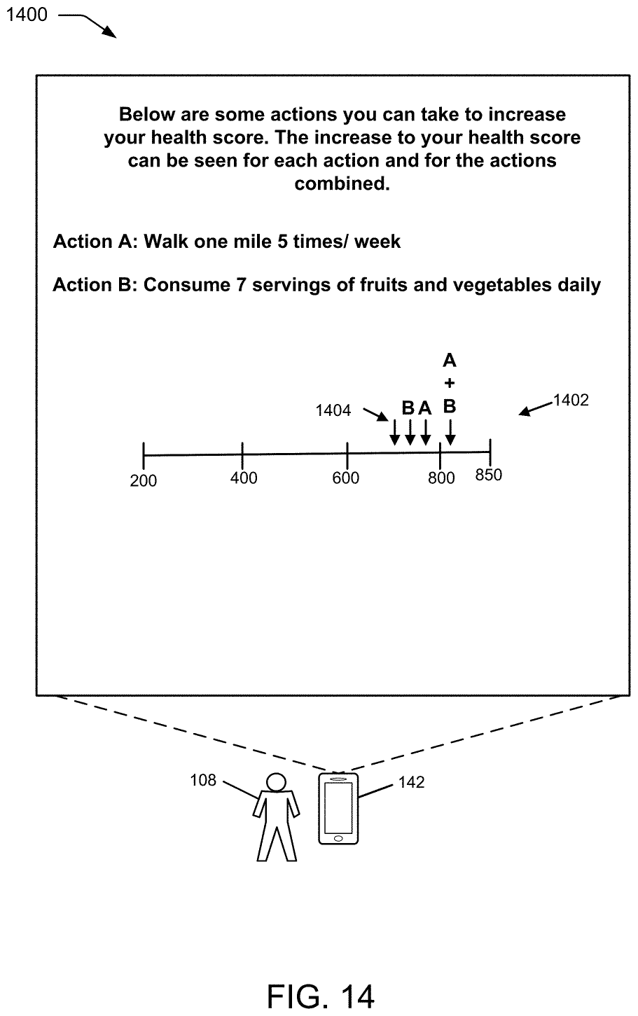

FIG. 14 is an example user interface 1400 including a recommendation to increase the health scores of an individual. The user interface 1400 can be displayed via the computing device 142 that is operated by the user 108. The user interface 1400 can be displayed by a mobile device app executed by the computing device 142 or a website accessed by the computing device 142. In additional implementations, the user interface 1400 can correspond to an email or text message received by the computing device 142. The user interface 1400 can be provided by a health science service provider, such as the health science servicer provider 102, to provide one or more recommendations to increase the health score of the individual 108.

The user interface 1400 can include text indicating a first recommendation with specific services opportunities, Recommendation with specific services opportunities A, and a second recommendation with specific services opportunities, Recommendation B with specific services opportunities, for increasing the health score of the individual 108. The user interface 1400 can also include a scale 1402 that shows a current health score 1404 of the individual 108. The user interface 1400 also includes indications of additional scores that can be obtained by the individual 108 based on the individual 108 taking action with respect to the first recommendation with specific services opportunities, the second recommendation with specific services opportunities, or both the first recommendation with specific services opportunities and the second recommendation with specific services opportunities. For example, in a situation where the individual 108 walks one mile five times per week for a specified period of time, the health score of the individual 108 can move to score A. In another example, in a scenario where the individual 108 consumes seven servings of fruits and vegetables daily for a specified period of time, the health score of the individual 108 can move to score B. In an additional example, in instances where the individual 108 walks one mile five times a week for a specified period of time and the individual consumes seven servings of fruits and vegetables daily for a specified period of time, the health score of the individual 108 can move to A+B. Thus, the user interface 1400 can indicate actions that the individual 108 can take to improve their health score and provide quantitative increases to health scores of the individual in situations where the individual takes one or more of the actions.

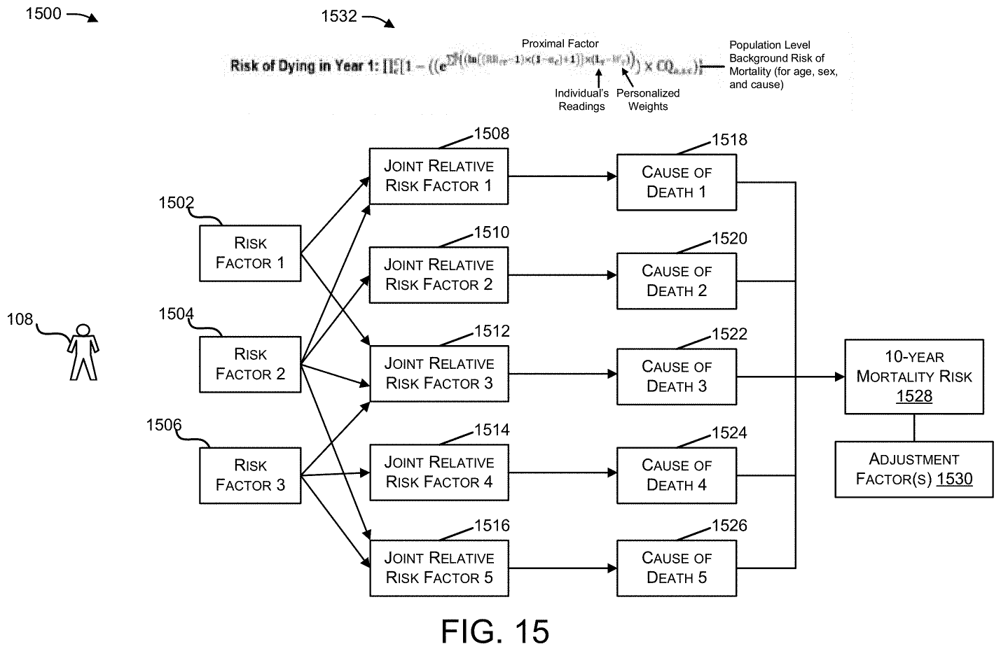

FIG. 15 illustrates a framework 1500 to determine a health indicator for an individual 108. The individual 108 can be associated with a number of risk factors for mortality, such as a first risk factor 1502, a second risk factor 1504, and a third risk factor 1506. In certain implementations, the risk factors 1502, 1504, 1506 can be associated with a joint risk for developing a particular cause of death. In the illustrative example of FIG. 15, the framework 1500 can include a first joint relative risk factor 1508 to which the first risk factor 1502 and the second risk factor 1504 contribute. Additionally, the framework 1500 can include a second joint relative risk factor 1510 to which the second risk factor 1504 contributes and a third relative risk factor 1512 to which the first risk factor 1502, the second risk factor 1504, the third risk factor 1506 contribute. Further, the framework 1500 can include a fourth joint relative risk factor 1514 to which the third risk factor 1506 contributes and a fifth joint relative risk factor 1516 to which the second risk factor 1504 and the third risk factor 1506 contribute.

The framework 1500 indicates that the first joint relative risk factor 1508 can correspond to a first cause of death 1518 and the second joint relative risk factor 1510 can correspond to a second cause of death 1520. In addition, the third joint relative risk factor 1512 can correspond to a third cause of death 1522 and the fourth joint relative risk factor 1514 can correspond to a fourth cause of death 1524. Further, the fifth joint relative risk factor 1516 can correspond to a fifth cause of death 1526. The relative risk of the individual 108 to develop the causes of death 1518, 1520, 1522, 1524, 1526 can be used to determine a health indicator of the individual 108, such as the 10-year mortality risk 1528. In certain implementations, the 10-year mortality risk 1528 can be modified based on one or more adjustment factors 1530. The one or more adjustment factors 1530 can include age and/or sex. In illustrative examples, the equation 1532 can be used in determining a health indicator of the individual 108.

Although various embodiments of the method and apparatus of the present invention have been illustrated herein in the Drawings and described in the Detailed Description, it will be understood that the invention is not limited to the embodiments disclosed, but is capable of numerous rearrangements, modifications and substitutions without departing from the scope of the present disclosure.

What is claimed is:

1. A system comprising:
one or more processors; and
one or more computer-readable storage media storing instructions that are executable by the one or more processors to perform operations comprising:
aggregating health data of an individual;
analyzing health indicator information to determine one or more factors that impact the health of the individual;
generating one or more models that include the one or more factors;
evaluating the one or more models with respect to the health data of the individual; and
determining one or more indicators of the health of the individual.

2. The system of claim 1, wherein the health data of the individual includes at least one of sensor data, genetic information, physical activity information, laboratory test data, medical imaging data, information included in medical records of the individual, or behavioral, nutritional and environmental data for the individual.

3. The system of claim 1, wherein the health indicator information includes at least one of clinical trials data, medical research information or other sources of data.

4. The system of claim 1, wherein the health indicator information includes one or more algorithms developed in conjunction with a health development platform.

5. The system of claim 1, wherein the operations further comprise:
validating the one or more models using additional health data from one or more populations.

6. A method comprising:
determining one or more indicators of the health of an individual;
determining one or more actions that the individual can take to increase at least one indicator of the one or more indicators;
determining an amount of increase in an indicator of the health of the individual for an action of the one or more actions;
determining that the amount of increase is above a threshold level; and
generating a recommendation with service opportunities including the action to increase the indicator of the health of the individual.

7. The method of claim 6, wherein determining one or more indicators of the health of an individual includes:
aggregating health data of a plurality of individuals;
determining a model indicating health of individuals, wherein the model includes a plurality of factors that impact the health of the individuals; and
evaluating the model with the health data of the plurality of individuals to generate one or more health indicators for each individual of the plurality of individuals.

8. The method of claim 6, further comprising:
determining that the indicator of the health of the individual changed; and
generating an additional recommendation with additional services opportunities based on a change to the indicator of the health of the individual.

9. The method of claim 6, further comprising:
generating a first user interface including the one or more indicators of the health of the individual;
generating a second user interface including the recommendation with the services opportunities to increase the indicator of the health of the individual; and
sending first data corresponding to the first user interface and second data corresponding to the second user interface to a computing device of the individual.

10. The method of claim 9, wherein the computing device of the individual is executing a health science application of a health science service provider and the first user interface and the second user interface are displayed in conjunction with the health science application.

11. A method comprising:
determining, by a health science service provider that includes one or more computing devices having one or more processors and memory, a plurality of health factors that have an impact on health of individuals;
generating, by the health science service provider, a model that includes the plurality of health factors;
performing, by the health science service provider, a validation process for the model;
collecting health data of an individual;
generating, by the health science service provider, a health indicator for the individual by applying the health data of the individual to the model;
determining, by the health science service provider, a subset of the plurality of health factors that have less than a threshold impact on the health indicator for the individual; and
determining, by the health science service provider, a recommendation for the individual based at least partly on the health indicator for the individual and the subset of the plurality of health factors, the recommendation including at least one action and at least one service.

12. The method of claim 11, wherein the at least one service is offered by a third-party service provider that is different from the health science service provider.

13. The method of claim 11, wherein the health science service provider determines the plurality of health factors based at least partly on analyzing at least one of medical literature, medical research studies, clinical trials, or scientific literature.

14. The method of claim 11, wherein the validation process for the model includes:
generating a performance metric for the model;
comparing the performance metric to at least one of one or more threshold performance metrics or a tolerance level for the performance metric; and.
modifying the model based at least partly on at least one of the performance metric being outside of a threshold performance metric or the performance metric being outside of the tolerance level for the performance metric.

15. The method of claim 11, wherein the validation process utilizes a dataset that is different from an additional dataset used to generate the model.

16. The method of claim 11, wherein the health indicator corresponds to an estimate of a mortality risk of the individual.

17. The method of claim 11, wherein the health indicator is determined based on a relative risk of the individual developing a cause of death, the relative risk comprising a combination of a first risk of developing the cause of death based on a first health factor of the plurality of health factors and a second risk of developing the cause of death based on a second health factor of the plurality of health factors.

18. The method of claim 11, further comprising determining an additional subset of the plurality of health factors that have greater than an additional threshold impact on the health indicator for the individual, the additional subset of the plurality of health factors being different from the subset of the plurality of health factors and the additional threshold impact being greater than the threshold impact.

19. The method of claim 11, wherein the recommendation is determined based at least partly on one or more behavioral characteristics of the individual.

20. The method of claim 11, further comprising:
obtaining additional health data of the individual that is collected subsequent to the health data of the individual;
generating an additional health indicator of the individual based at least partly on the additional health data; and
generating an additional recommendation for the individual based at least partly on the additional health indicator, the additional recommendation including an additional action and an addition service.
